(19) United States
(12) Patent Application Publication
(10) Pub. No.: US 2021/0079422 A1
(43) Pub. Date: Mar. 18, 2021

(54) AAV VECTOR COLUMN PURIFICATION METHODS

(71) Applicant: Spark Therapeutics, Inc., Philadelphia, PA (US)

(72) Inventors: Younghoon OH, Pennsylvania, PA (US); Guang QU, Sicklerville, NJ (US)

(73) Assignee: Spark Therapeutics, Inc., Philadelphia, PA (US)

(21) Appl. No.: 16/627,227

(22) PCT Filed: Jun. 29, 2018
(86) PCT No.: PCT/US18/40430

§ 371 Date: Dec. 27, 2019

Related U.S. Application Data
(60) Provisional application No. 62/527,633, filed on Jun. 30, 2017.
(60) Provisional application No. 62/531,744, filed on Jul. 12, 2017.
(60) Provisional application No. 62/567,905, filed on Oct. 4, 2017.

(51) Int. Cl. C12N 15/86; B01D 15/34; B01D 15/36

(52) U.S. Cl. CPC C12N 15/86; B01D 15/34; C12N 2750/14151; B01D 15/363; C12N 2750/14143; B01D 15/362

(57) ABSTRACT

Described and provided herein are purification, production and manufacturing methods for recombinant adeno-associated viral (rAAV) vector particles. Purification, production and manufacturing methods set forth herein, for example, include at least 2 column chromatography steps. Column chromatography steps include, for example, cation exchange chromatography, anion exchange chromatography, size exclusion chromatography and/or AAV affinity chromatography alone or in combination and in any order.

### RELATED APPLICATIONS

This patent application is the National Phase of International Application No. PCT/US2018/040430, filed Jun. 29, 2018, which designated the U.S. and that International Application was published under PCT Article 21(2) in English, which claims the benefit of priority to U.S. patent application No. 62/527,633, filed Jun. 30, 2017; U.S. patent application No. 62/531,744, filed Jul. 12, 2017; and U.S. patent application No. 62/567,905, filed Oct. 4, 2017. The entire contents of the foregoing applications are incorporated herein by reference, including all text, tables, drawings and sequences.

### SEQUENCE LISTING

The instant application contains a Sequence Listing which has been submitted electronically in ASCII format and is hereby incorporated by reference in its entirety. Said ASCII copy, created on Dec. 27, 2019, is named “Spark0509060_ST25.txt” and is 12.8 KB in size.

### INTRODUCTION

Gene delivery is a promising method for the treatment of acquired and inherited diseases. A number of viral-based systems for gene transfer purposes have been described, including adeno-associated virus (AAV)-based systems.

AAV is a helper-dependent DNA parvovirus that belongs to the genus Dependovirus. AAV requires helper virus function, e.g., adenovirus, herpes virus, or vaccinia, in order for a productive infection to occur. In the absence of a helper virus functions, AAV establishes a latent state by inserting its genome into a host cell chromosome. Subsequent infection by a helper virus rescues the integrated viral genome, which can then replicate to produce infectious AAV progeny.

AAV has a wide host range and is able to replicate in cells from any species in the presence of a suitable helper virus. For example, human AAV will replicate in canine cells co-infected with a canine adenovirus. AAV has not been associated with any human or animal disease and does not appear to adversely affect the biological properties of the host cell upon integration.

AAV vectors can be engineered to carry a heterologous nucleic acid sequence of interest (e.g., a selected gene encoding a therapeutic protein, an inhibitory nucleic acid such as an antisense molecule, a ribozyme, a miRNA, etc.) by deleting, in whole or in part, the internal portion of the AAV genome and inserting the nucleic acid sequence of interest between the ITRs. The ITRs remain functional in such vectors allowing replication and packaging of the rAAV containing the heterologous nucleic acid sequence of interest. The heterologous nucleic acid sequence is also typically linked to a promoter sequence capable of driving expression of the nucleic acid in the patient's target cells. Termination signals, such as polyadenylation sites, can also be included in the vector.

The construction of infectious recombinant AAV (rAAV) vectors has been described in a number of publications. See, e.g., U.S. Pat. Nos. 5,173,414 and 5,139,941; International Publication Numbers WO 92/01070 and WO 93/03769; Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; and Kotin, R. M. (1994) Human Gene Therapy 5:793-801.

Recombinant adeno-associated virus (AAV) vectors have shown excellent therapeutic promise in several early phase clinical trials by multiple groups. Development of this new class of biologic product towards approval will involve improvements in vector characterization and quality control methods, including a better understanding of how vector design and manufacturing process parameters affect impurity profiles in clinical grade vectors.

An important objective in the design of rAAV production and purification systems is to implement strategies to minimize/control the generation of production related impurities such as proteins, nucleic acids, and vector-related impurities, including wild-type/pseudo wild-type AAV species (wtAAV) and AAV-encapsidated residual DNA impurities. Removal of impurities in AAV vectors is complicated due to the way rAAV vectors are produced. In one production process, rAAV vectors are produced by a transient transfection process using three plasmids. Significant amounts of plasmid DNA are introduced into the cells to produce rAAV vectors. In addition, when rAAV vectors are released from the producing cells, cellular proteins and nucleic acids are co-released. Considering that the rAAV vector represents only about 1% of the biomass, it is very challenging to purify rAAV vectors to a level of purity which can be used as a clinical human gene therapy product. (Smith P H Wright J F. Qu G. et al 2003, Mo. Therapy, 7:8348; Chadeuf G. et al, Mo. Therapy 2005, 12:744. Report from the CHMP gene therapy expert group meeting. European Medicines Agency EMEA/CHMP 2005, 183989/2004).

Development of manufacturing processes to purify recombinant AAV as a product to treat human disease should achieve the following objectives: 1) consistent vector purity, potency and safety; 2) manufacturing process scalability; and 3) acceptable cost of manufacturing. Current ‘industry standard’ scalable AAV vector purification processes do not adequately achieve removal of impurities, which is important to meet the first objective listed above (consistent vector purity, potency and safety). Moreover, failure to adequately remove impurities using current industry-standard scalable purification processes has occurred because: 1) development of purification processes of viral products such as recombinant AAV for applications other than vaccines (in which an immune response is typically sought rather than avoided) is relatively new; 2) many groups involved in the development of scalable purification processes for AAV vectors have been unaware of the high levels of vector-related impurities and/or have assumed that such impurities will not contribute to a clinically significant vector immunogenicity; and 3) it is technically challenging to develop scalable purification processes suitable for the industry scale manufacture of rAAV vectors.

### SUMMARY

The invention provides purification and production methods for recombinant adeno-associated viral (rAAV) vector particles. The invention methods include at least 2 column chromatography steps.

In one embodiment, a method includes the steps of: (a) harvesting cells and/or cell culture supernatant comprising rAAV vector particles to produce a harvest; (b) optionally concentrating the harvest produced in step (a) to produce a concentrated harvest; (c) lysing the harvest produced in step (a) or the concentrated harvest produced in step (b) to produce a lysate; (d) treating the lysate produced in step (c) to reduce contaminating nucleic acid in the lysate thereby producing a nucleic acid reduced lysate; (e) optionally filtering the nucleic acid reduced lysate produced in step (d) to produce a clarified lysate, and optionally diluting the clarified lysate to produce a diluted clarified lysate; (f) subjecting the nucleic acid reduced lysate in in step (d), clarified lysate in step (e) or diluted clarified lysate produced in step (e) to cation exchange column chromatography to produce a column eluate comprised of rAAV vector particles thereby separating rAAV vector particles from protein impurities or other production/process related impurities, and optionally diluting the column eluate to produce a diluted column eluate; (g) subjecting the column eluate or the diluted column eluate produced in step (f) to anion exchange chromatography to produce a second column eluate comprised of rAAV vector particles thereby separating rAAV vector particles from protein impurities or production/process related impurities, and optionally concentrating the second column eluate to produce a concentrated second column eluate; (h) subjecting the second column eluate or the concentrated second column eluate produced in step (g) to size exclusion column chromatography (SEC) to produce a third column eluate comprised of rAAV vector particles thereby separating rAAV vector particles from protein impurities or production/process related impurities, and optionally concentrating the third column eluate to produce a concentrated third column eluate; and (i) filtering the third column eluate or the concentrated third column eluate produced in step (h) thereby producing purified rAAV vector particles.

In another embodiment, a method includes the steps of: (a) harvesting cells and/or cell culture supernatant comprising rAAV vector particles to produce a harvest; (b) optionally concentrating the harvest produced in step (a) to produce a concentrated harvest; (c) lysing the harvest produced in step (a) or the concentrated harvest produced in step (b) to produce a lysate; (d) treating the lysate produced in step (c) to reduce contaminating nucleic acid in the lysate thereby producing a nucleic acid reduced lysate; (e) optionally filtering the nucleic acid reduced lysate produced in step (d) to produce a clarified lysate, and optionally diluting the clarified lysate to produce a diluted clarified lysate; (f) subjecting the nucleic acid reduced lysate in in step (d), clarified lysate in step (e) or diluted clarified lysate produced in step (e) to cation exchange column chromatography to produce a column eluate comprised of rAAV vector particles thereby separating rAAV vector particles from protein impurities or other production/process related impurities, and optionally concentrating the column eluate to produce a concentrated column eluate; (g) subjecting the column eluate or the concentrated column eluate produced in step (f) to size exclusion column chromatography (SEC) to produce a second column eluate comprised of rAAV vector particles thereby separating rAAV vector particles from protein impurities or other production/process related impurities, and optionally diluting the second column eluate to produce a concentrated second column eluate; (h) subjecting the second column eluate or the diluted second column eluate produced in step (g) to anion exchange chromatography to produce a third column eluate comprised of rAAV vector particles thereby separating rAAV vector particles from protein impurities production/process related impurities, and optionally diluting the third column eluate to produce a diluted third column eluate; and (i) filtering the third column eluate or the concentrated third column eluate produced in step (h) thereby producing purified rAAV vector particles.

In a further embodiment, a method includes the steps of: (a) harvesting cells and/or cell culture supernatant comprising rAAV vector particles to produce a harvest; (b) optionally concentrating the harvest produced in step (a) to produce a concentrated harvest; (c) lysing the harvest produced in step (a) or the concentrated harvest produced in step (b) to produce a lysate; (d) treating the lysate produced in step (c) to reduce contaminating nucleic acid in the lysate thereby producing a nucleic acid reduced lysate; (e) optionally filtering the nucleic acid reduced lysate produced in step (d) to produce a clarified lysate, and optionally diluting the clarified lysate to produce a diluted clarified lysate; (f) subjecting the nucleic acid reduced lysate in in step (d), clarified lysate in step (e) or diluted clarified lysate produced in step (e) to cation exchange column chromatography to produce a column eluate comprised of rAAV vector particles thereby separating rAAV vector particles from protein impurities or other production/process related impurities, and optionally diluting the column eluate to produce a diluted column eluate; (g) subjecting the column eluate or the diluted column eluate produced in step (f) to anion exchange chromatography to produce a second column eluate comprised of rAAV vector particles thereby separating rAAV vector particles from production/process related impurities, and optionally concentrating the second column eluate to produce a concentrated second column eluate; (h) filtering the second column eluate or the concentrated second column eluate produced in step (g) thereby producing purified rAAV vector particles.

In an additional embodiment, a method includes the steps of: (a) harvesting cells and/or cell culture supernatant comprising rAAV vector particles to produce a harvest; (b) optionally concentrating the harvest produced in step (a) to produce a concentrated harvest; (c) lysing the harvest produced in step (a) or the concentrated harvest produced in step (b) to produce a lysate; (d) treating the lysate produced in step (c) to reduce contaminating nucleic acid in the lysate thereby producing a nucleic acid reduced lysate; (e) optionally filtering the nucleic acid reduced lysate produced in step (d) to produce a clarified lysate, and optionally diluting the clarified lysate to produce a diluted clarified lysate; (f) subjecting the nucleic acid reduced lysate in step (d), or clarified lysate or diluted clarified lysate produced in step (e) to AAV affinity column chromatography to produce a column eluate comprised of rAAV vector particles thereby separating rAAV vector particles from protein impurities or other production/process related impurities, and optionally diluting the column eluate to produce a diluted column eluate; (g) subjecting the column eluate or the diluted column eluate produced in step (f) to anion exchange chromatography to produce a second column eluate comprised of rAAV vector particles thereby separating rAAV vector particles from protein impurities or other production/process related impurities, and optionally concentrating the second column eluate to produce a concentrated second column eluate; (h) optionally subjecting the second column eluate or the concentrated second column eluate produced in step (g) to size exclusion column chromatography (SEC) to produce a third column eluate comprised of rAAV vector particles thereby separating rAAV vector particles from protein impurities or other production/process related impurities, and optionally concentrating the third column eluate to produce a concentrated third column eluate; and (i) filtering the second column eluate or the diluted second column eluate produced in step (g), or filtering the third column eluate or the concentrated third column eluate produced in step (h), thereby producing purified rAAV vector particles.

In yet another embodiment, a method includes the steps of: (a) harvesting cells and/or cell culture supernatant comprising rAAV vector particles to produce a harvest; (b) optionally concentrating the harvest produced in step (a) to produce a concentrated harvest; (c) lysing the harvest produced in step (a) or the concentrated harvest produced in step (b) to produce a lysate; (d) treating the lysate produced in step (c) to reduce contaminating nucleic acid in the lysate thereby producing a nucleic acid reduced lysate; (e) optionally filtering the nucleic acid reduced lysate produced in step (d) to produce a clarified lysate, and optionally diluting the clarified lysate to produce a diluted clarified lysate; (f) subjecting the nucleic acid reduced lysate in step (d), or clarified lysate or diluted clarified lysate produced in step (e) to AAV affinity column chromatography to produce a column eluate comprised of rAAV vector particles thereby separating rAAV vector particles from protein impurities or other production/process related impurities, and optionally concentrating the column eluate to produce a concentrated column eluate; (g) subjecting the column eluate or the concentrated column eluate produced in step (f) to size exclusion column chromatography (SEC) to produce a second column eluate comprised of rAAV vector particles thereby separating rAAV vector particles from protein impurities or other production/process related impurities, and optionally diluting the second column eluate to produce a diluted second column eluate; (h) optionally subjecting the second column eluate or the diluted second column eluate produced in step (g) to anion exchange chromatography to produce a third column eluate comprised of rAAV vector particles thereby separating rAAV vector particles from protein impurities or other production/process related impurities, and optionally diluting the third column eluate to produce a diluted third column eluate; and (i) filtering the second column eluate or the diluted second column eluate produced in step (g), or filtering the third column eluate or the concentrated third column eluate produced in step (h), thereby producing purified rAAV vector particles.

In particular aspects of the invention methods, concentrating of step (b) and/or step (f) and/or step (g) and/or step (h) is via ultrafiltration/diafiltration, such as by tangential flow filtration (TFF).

In particular aspects of the invention methods, concentrating of step (b) reduces the volume of the harvested cells and cell culture supernatant by about 2-20 fold.

In particular aspects of the invention methods, concentrating of step (f) and/or step (g) and/or step (h) reduces the volume of the column eluate by about 5-20 fold.

In particular aspects of the invention methods, lysing of the harvest produced in step (a) or the concentrated harvest produced in step (b) is by physical or chemical means. Non-limiting examples of physical means include microfluidization and homogenization. Non-limiting examples of chemical means include detergents. Detergents include non-ionic and ionic detergents. Non limiting examples of non-ionic detergents include triton X-100. Non limiting examples of detergent concentration is between about 0.1 and 1.0%, inclusive.

In particular aspects of the invention methods, step (d) comprises treating with a nuclease thereby reducing contaminating nucleic acid. Non limiting examples of a nuclease include benzonase.

In particular aspects of the invention methods, filtering of the clarified lysate or the diluted clarified lysate of step (e) is via a filter. Non limiting examples of filters are those having a pore diameter of between about 0.1 and 10.0 microns, inclusive.

In particular aspects of the invention methods, diluting of the clarified lysate of step (e) is with an aqueous buffered phosphate, acetate or Tris solution. Non limiting examples of solution pH are between about 4.0 and 7.4, inclusive. Non limiting examples of Tris solution pH are greater than 7.5, such as between about 8.0 and 9.0, inclusive.

In particular aspects of the invention methods, diluting of the column eluate of step (f) or the second column eluate of step (g) is with an aqueous buffered phosphate, acetate or Tris solution. Non limiting examples of solution pH are between about 4.0 and 7.4, inclusive. Non limiting examples of Tris solution pH are greater than 7.5, such as between about 8.0 and 9.0, inclusive.

In particular aspects of the invention methods, the rAAV vector particles resulting from step (i) are formulated with a surfactant to produce an AAV vector formulation.

In particular aspects of the invention methods, an anion exchange column chromatography of step (f), (g) and/or (h) comprises polyethylene glycol (PEG) modulated column chromatography.

In particular aspects of the invention methods, an anion exchange column chromatography of step (g) and/or (h) is washed with a PEG solution prior to elution of the rAAV vector particles from the column.

In particular aspects of the invention methods, PEG has an average molecular weight in a range of about 1,000 to 80,000 g/mol, inclusive.

In particular aspects of the invention methods, PEG is at a concentration of about 4% to about 10%, inclusive.

In particular aspects of the invention methods, an anion exchange column of step (g) and/or (h) is washed with an aqueous surfactant solution prior to elution of the rAAV vector particles from the column.

In particular aspects of the invention methods, a cation exchange column of step (f) is washed with a surfactant solution prior to elution of the rAAV vector particles from the column.

In particular aspects of the invention methods, a PEG solution and/or the surfactant solution comprises an aqueous Tris-Cl/NaCl buffer, an aqueous phosphate/NaCl buffer or an aqueous acetate/NaCl buffer.

In particular aspects of the invention methods, NaCl in a buffer or solution is in a range of between about 20-300 mM NaCl, inclusive, or between about 50-250 mM NaCl, inclusive.

In particular aspects of the invention methods, a surfactant comprises a cationic or anionic surfactant.

In particular aspects of the invention methods, a surfactant comprises a twelve carbon chained surfactant.

In particular aspects of the invention methods, a surfactant comprises Dodecyltrimethylammonium chloride (DTAC) or Sarkosyl.

In particular aspects of the invention methods, rAAV vector particles are eluted from the anion exchange column of step (f), (g) and/or (h) with an aqueous Tris-Cl/NaCl buffer.

In particular aspects of the invention methods, a Tris-Cl/NaCl buffer comprises 100-400 mM NaCl, inclusive, optionally at a pH in a range of about 7.5 to about 9.0, inclusive.

In particular aspects of the invention methods, the anion exchange column of step (f), (g) and/or (h) is washed with an aqueous Tris-Cl/NaCl buffer.

In particular aspects of the invention methods, NaCl in an aqueous Tris-Cl/NaCl buffer is in a range of about 75-125 mM, inclusive.

In particular aspects of the invention methods, an aqueous Tris-Cl/NaCl buffer has a pH from about 7.5 to about 9.0, inclusive.

In particular aspects of the invention methods, an anion exchange column of step (f), (g) and/or (h) is washed one or more times to reduce the amount of AAV empty capsids in the second or third column eluate.

In particular aspects of the invention methods, an anion exchange column wash removes AAV empty capsids from the column prior to rAAV removal and/or instead of rAAV, thereby reducing the amount of AAV empty capsids in the second or third column eluate.

In particular aspects of the invention methods, an anion exchange column wash removes at least about 50% of the total AAV empty capsids from the column prior to rAAV removal and/or instead of rAAV, thereby reducing the amount of AAV empty capsids in the second or third column eluate by about 50%.

In particular aspects of the invention methods, NaCl in the aqueous Tris-Cl/NaCl buffer is in a range of about 110-120 mM, inclusive.

In particular aspects of the invention methods, ratios and/or amounts of the rAAV vector particles and AAV empty capsids eluted are controlled by a wash buffer.

In particular aspects of the invention methods, the vector particles are eluted from the cation exchange column of step (f) in an aqueous phosphate/NaCl buffer or an aqueous acetate/NaCl buffer. Non limiting NaCl concentration in a buffer is in a range of about 125-500 mM NaCl, inclusive. Non limiting examples of buffer pH are between about 5.5 to about 7.5, inclusive.

In particular aspects of the invention methods, an anion exchange column of step (f), (g) and/or (h) comprises a quarternary ammonium functional group such as quaternized polyethyleneimine.

In particular aspects of the invention methods, a size exclusion column (SEC) of step (g) and/or (h) has a separation/fractionation range (Molecular weight) from about 10,000 to about 600,000, inclusive.

In particular aspects of the invention methods, a cation exchange column of step (f) comprises a sulfonic acid or functional group such as sulphopropyl.

In particular aspects of the invention methods, an AAV affinity column comprises a protein or ligand that binds to AAV capsid protein. Non-limiting examples of a protein include an antibody that binds to AAV capsid protein. More specific non-limiting examples include a single-chain Llama antibody (Camelid) that binds to AAV capsid protein.

In particular aspects of the invention methods, a method excludes a step of cesium chloride gradient ultracentrifugation.

In particular aspects of the invention methods, rAAV vector particles comprise a transgene that encodes a nucleic acid selected from the group consisting of a siRNA, an antisense molecule, miRNA a ribozyme and a shRNA.

In particular aspects of the invention methods, rAAV vector particles comprise a transgene that encodes a gene product selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), TGFβ, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase.

In particular aspects of the invention methods, rAAV vector particles comprise a transgene that encodes a gene product selected from the group consisting of thrombopoietin (TPO), interleukins (IL1 through IL-17), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

In particular aspects of the invention methods, the rAAV vector particles comprise a transgene encoding a protein useful for correction of in born errors of metabolism selected from the group consisting of carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor V, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, RPE65, H-protein, T-protein, a cystic fibrosis transmembrane regulator (CFTR) sequence, and a dystrophin cDNA sequence.

In particular aspects of the invention methods, the rAAV vector particles comprise a transgene that encodes Factor VIII or Factor IX.

In particular aspects of the invention methods, a method recovers approximately 50-90% of the total rAAV vector particles from the harvest produced in step (a) or the concentrated harvest produced in step (b).

In particular aspects of the invention methods, a method produces rAAV vector particles having a greater purity than rAAV vector particles produced or purified by a single AAV affinity column purification.

In particular aspects of the invention methods, steps (c) and (d) are performed substantially concurrently.

In particular aspects of the invention methods, NaCl is adjusted to be in a range of about 100-400 mM NaCl, inclusive, or in a range of about 140-300 mM NaCl, inclusive, after step (c) but prior to step (f).

In particular aspects of the invention methods, rAAV vector particles are derived from an AAV selected from the group consisting of AAV1, AAV2, AAV3, AAV4, AAV5, AAV6, AAV7, AAV8, AAV9, AAV10, Rh10 and Rh74.

In particular aspects of the invention methods, rAAV vector particles comprise a capsid sequence having 70% or more identity to an AAV1, AAV2, AAV3, AAV4, AAV5, AAV6, AAV7, AAV8, AAV9, AAV10, Rh10, Rh74, SEQ ID NO:1 or SEQ ID NO:2 capsid sequence.

In particular aspects of the invention methods, rAAV vector particles comprise an ITR sequence having 70% or more identity to an AAV1, AAV2, AAV3, AAV4, AAV5, AAV6, AAV7, AAV8, AAV9, AAV10, Rh10, or Rh74 ITR sequence.

In particular aspects of the invention methods, cells are suspension or adherent cells.

In particular aspects of the invention methods, cells are mammalian cells. Non-limiting examples include HEK cells, such as HEK-293 cells.

In particular aspects of the invention methods, a method is performed according to any one or more column, condition, concentration, molarity, volume, capacity, flow rate, pressure, material, temperature, pH, or step as set forth in any of Examples 1-3.

In particular aspects of the invention methods, cell lysis and/or preparation prior to column purification as set forth herein is performed according to any one or more condition, concentration, molarity, volume, capacity, flow rate, pressure, material, temperature, pH, or step as set forth in Example 4.

### DESCRIPTION OF DRAWINGS

FIG. 1 shows CEX (Poros 50HS)→AEX (Poros 50HQ)>UF>SEC (Superdex 200 prep grade) column chromatography of a 500-600 ml starting rAAV harvest volume that can be scaled up to larger volumes for substantially increased rAAV production (e.g., 1.2 L or larger).

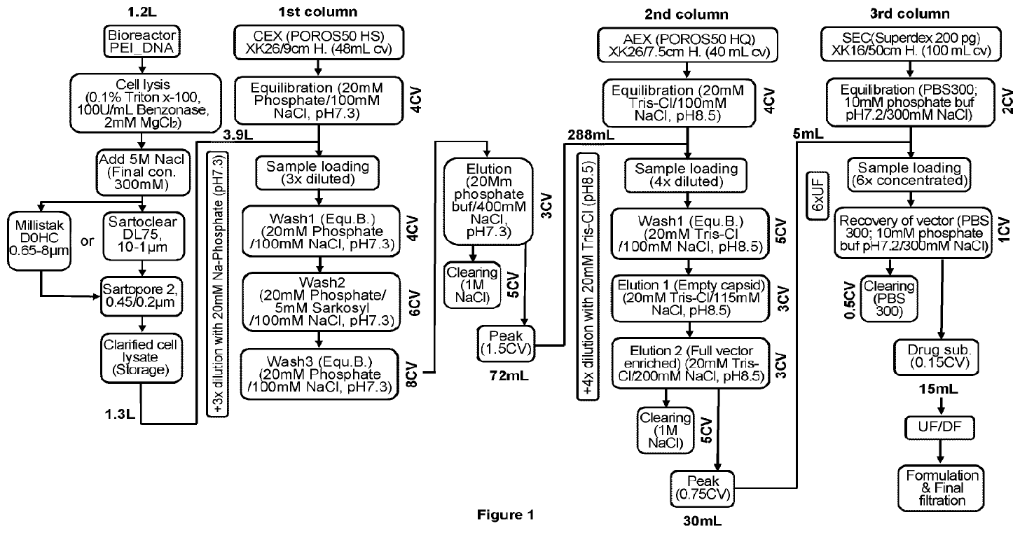

FIG. 2 shows CEX (Poros 50HS)→AEX (Poros 50HQ) column chromatography of a 500-600 ml starting rAAV harvest volume that can be scaled up to larger volumes for substantially increased rAAV production (e.g., 1.2 L or larger).

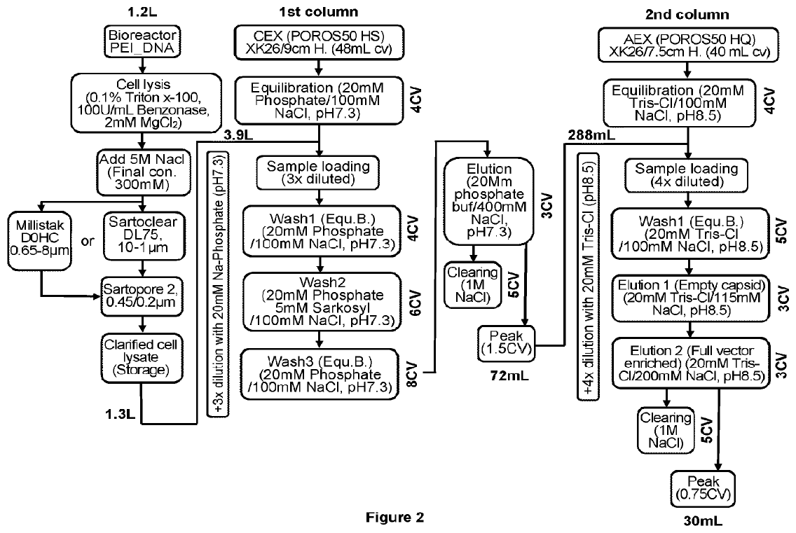

FIG. 3 shows CEX (Poros 50HS)→UF>SEC (Superdex 200 prep grade)>AEX (Poros 50HQ) column chromatography of a 500-600 ml starting rAAV harvest volume that can be scaled up to larger volumes for substantially increased rAAV production (e.g., 1.2 L or larger).

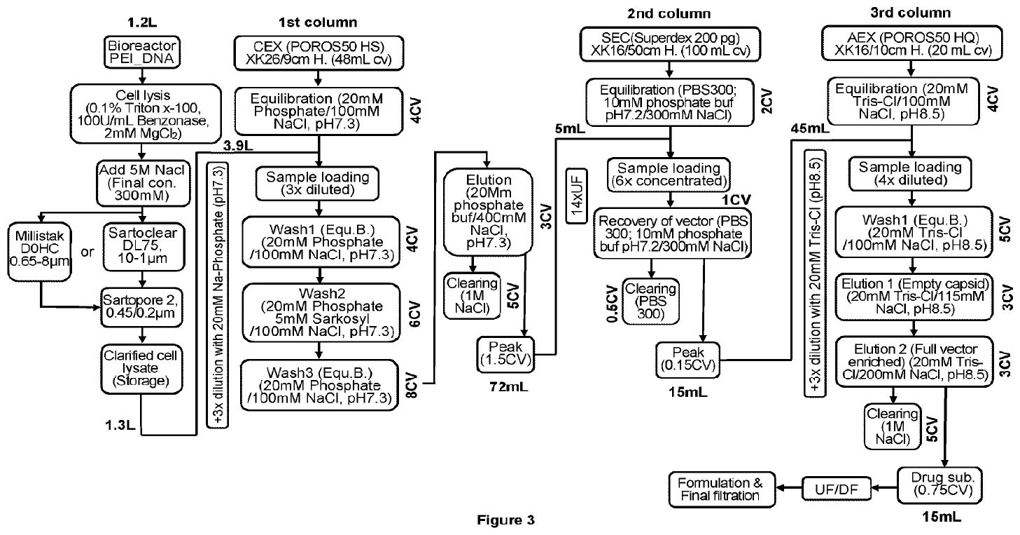

FIG. 4 shows Affinity (AVB Sepharose HP)→AEX (Poros 50HQ) column chromatography of a 500-600 ml starting rAAV harvest volume that can be scaled up to larger volumes for substantially increased rAAV production (e.g., 1.2 L or larger). After a low pH elution from the affinity column (about 7.5 mL eluate), 4.5 mL of a high pH neutralization solution is added followed by adding about 120 mL of 5 mM Tris-Cl pH 8.5)/40 mM NaCl solution for a total load volume of about 132 mL for the AEX (Poros 50HQ) column.

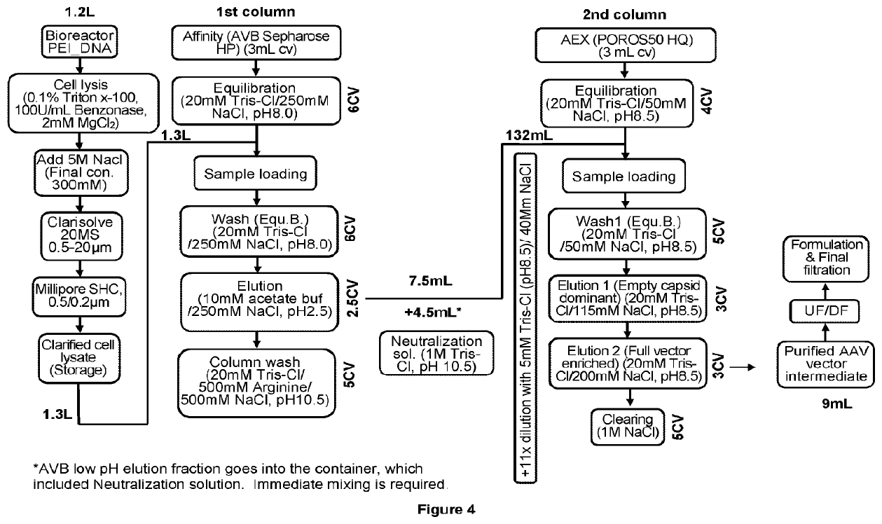

### DETAILED DESCRIPTION

The invention provides a recombinant adeno-associated virus (AAV) vector (rAAV) vector purification and production methods that are scalable up to large scale. For example a suspension culture 5, 10, 10-20, 20-50, 50-100, 100-200 or more liters volume. The invention provides recombinant adeno-associated virus (AAV) vector (rAAV) vector purification and production methods that are also applicable to a wide variety of AAV serotypes/capsid variants. The invention methods used for purification or production of rAAV vector include removal of in process impurities and in production related impurities. The invention methods involve a unique combination of chromatography steps and process steps that provides scalability to purify many different serotypes/pseudotypes of rAAV vectors.

Impurities include AAV vector production related impurities which include proteins, nucleic acids (e.g., DNA), cellular components such as intracellular and membrane components which are impurities distinct from the AAV vectors. The term “production or process related impurities” refers to any components released during the AAV purification and production process that are not bona fide rAAV particles.

Bona fide rAAV vectors refer to rAAV vector particles comprising the heterologous nucleic acid (e.g., transgene) which are capable of infecting target cells. The phrase excludes empty AAV capsids, AAV vectors lacking full inserts in the packaged genome or AAV vectors containing contaminating host cell nucleic acids. In certain embodiments, bona fide rAAV vectors refer to rAAV vector particles that also lack contaminating plasmid sequences in the packaged vector genome.

“Empty capsids” and “empty particles” refer to an AAV particle or virion that includes an AAV capsid shell but that lacks in whole or part the genome comprising the heterologous nucleic acid sequence flanked on one or both sides by AAV ITRs. Such empty capsids do not function to transfer the heterologous nucleic acid sequence into the host cell or cells within an organism.

The term “vector” refers to small carrier of nucleic acid molecule, a plasmid, virus (e.g., rAAV vector), or other vehicle that can be manipulated by insertion or incorporation of a nucleic acid. Vectors can be used for genetic manipulation (i.e., “cloning vectors”), to introduce/transfer polynucleotides into cells, and to transcribe or translate the inserted polynucleotide in cells. An “expression vector” is a vector that contains a gene or nucleic acid sequence with the necessary regulatory regions needed for expression in a host cell. A vector nucleic acid sequence generally contains at least an origin of replication for propagation in a cell and optionally additional elements, such as a heterologous nucleic acid sequence, expression control element (e.g., a promoter, enhancer), intron, inverted terminal repeats (ITRs), optional selectable marker, polyadenylation signal.

A rAAV vector is derived from adeno-associated virus. AAV vectors are useful as gene therapy vectors as they can introduce nucleic acid/genetic material into cells so that the nucleic acid/genetic material may be maintained in cells. Because AAV are not associated with pathogenic disease in humans, rAAV vectors are able to deliver heterologous nucleic acid sequences (e.g., therapeutic proteins and agents) to human patients without causing substantial AAV pathogenesis or disease.

The term “recombinant,” as a modifier of vector, such as rAAV vectors, as well as a modifier of sequences such as recombinant polynucleotides and polypeptides, means that the compositions have been manipulated (i.e., engineered) in a fashion that generally does not occur in nature. A particular example of a recombinant AAV vector would be where a nucleic acid that is not normally present in the wild-type AAV genome is inserted within the viral genome. An example of would be where a nucleic acid (e.g., gene) encoding a therapeutic protein or polynucleotide sequence is cloned into a vector, with or without 5′, 3′ and/or intron regions that the gene is normally associated within the AAV genome. Although the term “recombinant” is not always used herein in reference to AAV vectors, as well as sequences such as polynucleotides, recombinant forms including AAV vectors, polynucleotides, etc., are expressly included in spite of any such omission.

A “rAAV vector” is derived from the wild type genome of a virus, such as AAV by using molecular methods to remove the wild type genome from AAV genome, and replacing with a non-native (heterologous) nucleic acid, such as a nucleic acid encoding a therapeutic protein or polynucleotide sequence. Typically, for AAV one or both inverted terminal repeat (ITR) sequences of AAV genome are retained in the rAAV vector. A rAAV is distinguished from an AAV genome since all or a part of the AAV genome has been replaced with a non-native sequence with respect to the AAV genomic nucleic acid, such as with a heterologous nucleic acid encoding a therapeutic protein or polynucleotide sequence. Incorporation of a non-native sequence therefore defines the AAV as a “recombinant” AAV vector, which can be referred to as a “rAAV vector.”

A recombinant AAV vector sequence can be packaged-referred to herein as a “particle” for subsequent infection (transduction) of a cell, ex vivo, in vitro or in vivo. Where a recombinant vector sequence is encapsidated or packaged into an AAV particle, the particle can also be referred to as a “rAAV” or “rAAV particle” or “rAAV virion.” Such rAAV, rAAV particles and rAAV virions include proteins that encapsidate or package the vector genome. Particular examples include in the case of AAV, capsid proteins.

A vector “genome” refers to the portion of the recombinant plasmid sequence that is ultimately packaged or encapsidated to form a rAAV particle. In cases where recombinant plasmids are used to construct or manufacture recombinant AAV vectors, the AAV vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsidated into rAAV particles. Thus, a vector “genome” refers to the nucleic acid that is packaged or encapsidated by rAAV.

“AAV helper functions” refer to AAV-derived coding sequences (proteins) which can be expressed to provide AAV gene products and AAV vectors that, in turn, function in trans for productive AAV replication and packaging. Thus, AAV helper functions include AAV open reading frames (ORFs), including rep and cap and others such as AAP for certain AAV serotypes. The Rep expression products have been shown to possess many functions, including, among others: recognition, binding and nicking of the AAV origin of DNA replication; DNA helicase activity; and modulation of transcription from AAV (or other heterologous) promoters. The Cap expression products (capsids) supply necessary packaging functions. AAV helper functions are used to complement AAV functions in trans that are missing from AAV vector genomes.

An “AAV helper construct” refers generally to a nucleic acid sequence that includes nucleotide sequences providing AAV functions deleted from an AAV vector which is to be used to produce a transducing AVV vector for delivery of a nucleic acid sequence of interest, by way of gene therapy to a subject, for example. AAV helper constructs are commonly used to provide transient expression of AAV rep and/or cap genes to complement missing AAV functions that are necessary for AAV vector replication. Helper constructs generally lack AAV ITRs and can neither replicate nor package themselves. AAV helper constructs can be in the form of a plasmid, phage, transposon, cosmid, virus, or virion. A number of AAV helper constructs have been described, such as plasmids pAAV/Ad and pIM29+45 which encode both Rep and Cap expression products (See, e.g., Samulski et al. (1989) J. Virol. 63:3822-3828; and McCarty et al. (1991) J. Virol. 65:2936-2945). A number of other vectors have been described which encode Rep and/or Cap expression products (See, e.g., U.S. Pat. Nos. 5,139,941 and 6,376,237).

The term “accessory functions” refers to non-AAV derived viral and/or cellular functions upon which AAV is dependent for replication. The term includes proteins and RNAs that are required in AAV replication, including moieties involved in activation of AAV gene transcription, stage specific AAV mRNA splicing, AAV DNA replication, synthesis of Cap expression products and AAV capsid packaging. Viral-based accessory functions can be derived from any of the known helper viruses such as adenovirus, herpesvirus (other than herpes simplex virus type-1) and vaccinia virus.

An “accessory function vector” refers generally to a nucleic acid molecule that includes polynucleotide sequences providing accessory functions. Such sequences can be on an accessory function vector, and transfected into a suitable host cell. The accessory function vector is capable of supporting rAAV virion production in the host cell. Accessory function vectors can be in the form of a plasmid, phage, transposon or cosmid. In addition, the full-complement of adenovirus genes are not required for accessory functions. For example, adenovirus mutants incapable of DNA replication and late gene synthesis have been reported to be permissive for AAV replication (Ito et al., (1970) J. Gen. Virol. 9:243; Ishibashi et al, (1971) Virology 45:317). Similarly, mutants within E2B and E3 regions have been shown to support AAV replication, indicating that the E2B and E3 regions are probably not involved in providing accessory functions (Carter et al., (1983) Virology 126:505). Adenoviruses defective in the E1 region, or having a deleted E4 region, are unable to support AAV replication. Thus, E1A and E4 regions appear necessary for AAV replication, either directly or indirectly (Laughlin et al., (1982) J. Virol. 41:868; Janik et al., (1981) Proc. Natl. Acad. Sci. USA 78:1925; Carter et al., (1983) Virology 126:505). Other characterized Adenovirus mutants include: E1B (Laughlin et al. (1982), supra; Janik et al. (1981), supra; Ostrove et al., (1980) Virology 104:502); E2A (Handa et al., (1975) J. Gen. Virol. 29:239; Strauss et al., (1976) J. Virol. 17:140; Myers et al., (1980) J. Virol. 35:665; Jay et al., (1981) Proc. Natl. Acad. Sci. USA 78:2927; Myers et al., (1981) J. Biol. Chem. 256:567); E2B (Carter, Adeno-Associated Virus Helper Functions, in I CRC Handbook of Parvoviruses (P. Tijssen ed., 1990)); E3 (Carter et al. (1983), supra); and E4 (Carter et al. (1983), supra; Carter (1995)). Studies of the accessory functions provided by adenoviruses having mutations in the E1B coding region have produced conflicting results, but E1B55k may be required for AAV virion production, while E1B19k is not (Samulski et al., (1988) J. Virol. 62:206-210). In addition, International Publication WO 97/17458 and Matshushita et al., (1998) Gene Therapy 5:938-945, describe accessory function vectors encoding various Adenovirus genes. Exemplary accessory function vectors comprise an adenovirus VA RNA coding region, an adenovirus E4 ORF6 coding region, an adenovirus E2A 72 kD coding region, an adenovirus E1A coding region, and an adenovirus E1B region lacking an intact E1B55k coding region. Such accessory function vectors are described, for example, in International Publication No. WO 01/83797.

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Cross-reactivity differences are usually due to differences in capsid protein sequences/antigenic determinants (e.g., due to VP1, VP2, and/or VP3 sequence differences of AAV serotypes).

Under the traditional definition, a serotype means that the virus of interest has been tested against serum specific for all existing and characterized serotypes for neutralizing activity and no antibodies have been found that neutralize the virus of interest. As more naturally occurring virus isolates of are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

rAAV vectors include any viral strain or serotype. As a non-limiting example, a rAAV plasmid or vector genome or particle (capsid) can be based upon any AAV serotype, such as AAV-1, -2, -3, -4, -5, -6, -7, -8, -9, -10, -11, for example. Such vectors can be based on the same of strain or serotype (or subgroup or variant), or be different from each other. As a non-limiting example, a rAAV plasmid or vector genome or particle (capsid) based upon one serotype genome can be identical to one or more of the capsid proteins that package the vector. In addition, a rAAV plasmid or vector genome can be based upon an AAV (e.g., AAV2) serotype genome distinct from one or more of the capsid proteins that package the vector genome, in which case at least one of the three capsid proteins could be a AAV1, AAV2, AAV3, AAV4, AAV5, AAV6, AAV7, AAV8, AAV9, AAV10, AAV11, Rh10, Rh74, SEQ ID NO:1 or SEQ ID NO:2 or variant thereof, for example. rAAV vectors therefore include gene/protein sequences identical to gene/protein sequences characteristic for a particular serotype, as well as mixed serotypes.

In various exemplary embodiments, a rAAV vector includes or consists of a capsid sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, AAV11, Rh10, Rh74, SEQ ID NO:1 or SEQ ID NO:2 capsid proteins. In various exemplary embodiments, a rAAV vector includes or consists of a sequence at least 70% or more (e.g., 75%, 80%, 85%, 90%, 95%, 96%, 97%, 98%, 99%, 99.5%, etc.) identical to one or more AAV1, AAV2, AAV3, AAV4, AAV5, AAV6, AAV7, AAV8, AAV9, AAV10, AAV11, Rh10, or Rh74 ITR(s).

rAAV, such as AAV1, AAV2, AAV3, AAV4, AAV5, AAV6, AAV7, AAV8, AAV9, AAV10, AAV11, Rh10, Rh74, SEQ ID NO:1 and SEQ ID NO:2 and variant, hybrid and chimeric sequences, can be constructed using recombinant techniques that are known to the skilled artisan, to include one or more heterologous polynucleotide sequences (transgenes) flanked with one or more functional AAV ITR sequences. Such vectors have one or more of the wild type AAV genes deleted in whole or in part, but retain at least one functional flanking ITR sequence(s), as necessary for the rescue, replication, and packaging of the recombinant vector into a rAAV vector particle. A rAAV vector genome would therefore include sequences required in cis for replication and packaging (e.g., functional ITR sequences)

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Nucleic acids include naturally occurring, synthetic, and intentionally modified or altered polynucleotides. Nucleic acids can be single, double, or triplex, linear or circular, and can be of any length. In discussing nucleic acids, a sequence or structure of a particular polynucleotide may be described herein according to the convention of providing the sequence in the 5′ to 3′ direction.

A “heterologous” nucleic acid sequence refers to a polynucleotide inserted into a AAV plasmid or vector for purposes of vector mediated transfer/delivery of the polynucleotide into a cell. Heterologous nucleic acid sequences are distinct from AAV nucleic acid, i.e., are non-native with respect to AAV nucleic acid. Once transferred/delivered into the cell, a heterologous nucleic acid sequence, contained within the vector, can be expressed (e.g., transcribed, and translated if appropriate). Alternatively, a transferred/delivered heterologous polynucleotide in a cell, contained within the vector, need not be expressed. Although the term “heterologous” is not always used herein in reference to nucleic acid sequences and polynucleotides, reference to a nucleic acid sequence or polynucleotide even in the absence of the modifier “heterologous” is intended to include heterologous nucleic acid sequences and polynucleotides in spite of the omission.

The “polypeptides,” “proteins” and “peptides” encoded by the “nucleic acid sequence,” include full-length native sequences, as with naturally occurring proteins, as well as functional subsequences, modified forms or sequence variants so long as the subsequence, modified form or variant retains some degree of functionality of the native full-length protein. Such polypeptides, proteins and peptides encoded by the nucleic acid sequences can be but are not required to be identical to the endogenous protein that is defective, or whose expression is insufficient, or deficient in the treated mammal.

A “transgene” is used herein to conveniently refer to a nucleic acid (e.g., heterologous) that is intended or has been introduced into a cell or organism. Transgenes include any nucleic acid, such as a heterologous nucleic acid encoding a therapeutic protein or polynucleotide sequence.

In a cell having a transgene, the transgene has been introduced/transferred by way of a plasmid or a AAV vector, “transduction” or “transfection” of the cell. The terms “transduce” and “transfect” refer to introduction of a molecule such as a nucleic acid into a host cell (e.g., HEK293) or cells of an organism. The transgene may or may not be integrated into genomic nucleic acid of the recipient cell. If an introduced nucleic acid becomes integrated into the nucleic acid (genomic DNA) of the recipient cell or organism it can be stably maintained in that cell or organism and further passed on to or inherited by progeny cells or organisms of the recipient cell or cells of an organism.

A “host cell” denotes, for example, microorganisms, yeast cells, insect cells, and mammalian cells, that can be, or have been, used as recipients of an AAV vector plasmid, AAV helper construct, an accessory function vector, or other transfer DNA. The term includes the progeny of the original cell which has been transfected. Thus, a “host cell” generally refers to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. Exemplary host cells include human embryonic kidney (HEK) cells such as HEK293.

A “transduced cell” is a cell into which a transgene has been introduced. Accordingly, a “transduced” cell means a genetic change in a cell following incorporation of an exogenous molecule, for example, a nucleic acid (e.g., a transgene) into the cell. Thus, a “transduced” cell is a cell into which, or a progeny thereof in which an exogenous nucleic acid has been introduced. The cell(s) can be propagated (cultured) and the introduced protein expressed or nucleic acid transcribed, or vector, such as rAAV, produced by the cell. For gene therapy uses and methods, a transduced cell can be in a subject.

As used herein, the term “stable” in reference to a cell, or “stably integrated” means that nucleic acid sequences, such as a selectable marker or heterologous nucleic acid sequence, or plasmid or vector has been inserted into a chromosome (e.g., by homologous recombination, non-homologous end joining, transfection, etc.) or is maintained in the recipient cell or host organism extrachromosomally, and has remained in the chromosome or is maintained extrachromosomally for a period of time. In the case of culture cells, nucleic acid sequences, such as a heterologous nucleic acid sequence, or plasmid or vector has been inserted into a chromosome can be maintained over the course of a plurality of cell passages.

A “cell line” refers to a population of cells capable of continuous or prolonged growth and division in vitro under appropriate culture conditions. Cell lines can, but need not be, clonal populations derived from a single progenitor cell. In cell lines, spontaneous or induced changes can occur in karyotype during storage or transfer of such clonal populations, as well as during prolonged passaging in tissue culture. Thus, progeny cells derived from the cell line may not be precisely identical to the ancestral cells or cultures. An exemplary cell line applicable to the invention purification methods is HEK293.

An “expression control element” refers to nucleic acid sequence(s) that influence expression of an operably linked nucleic acid. Control elements, including expression control elements as set forth herein such as promoters and enhancers. rAAV vectors can include one or more “expression control elements.” Typically, such elements are included to facilitate proper heterologous polynucleotide transcription and if appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but may also act in trans.

Expression control can be effected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances. Nevertheless, owing to the length limitations of rAAV vectors, expression control elements will typically be within 1 to 1000 nucleotides from the transcribed nucleic acid.

Functionally, expression of operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed sequence. A promoter typically increases an amount expressed from operably linked nucleic acid as compared to an amount expressed when no promoter exists.

An “enhancer” as used herein can refer to a sequence that is located adjacent to the nucleic acid sequence, such as selectable marker, or heterologous nucleic acid sequence Enhancer elements are typically located upstream of a promoter element but also function and can be located downstream of or within a sequence. Hence, an enhancer element can be located upstream or downstream, e.g., within 100 base pairs, 200 base pairs, or 300 or more base pairs of the as selectable marker, and/or a heterologous nucleic acid encoding a therapeutic protein or polynucleotide sequence. Enhancer elements typically increase expression of an operably linked nucleic acid above expression afforded by a promoter element.

The term “operably linked” means that the regulatory sequences necessary for expression of a nucleic acid sequence are placed in the appropriate positions relative to the sequence so as to effect expression of the nucleic acid sequence. This same definition is sometimes applied to the arrangement of nucleic acid sequences and transcription control elements (e.g. promoters, enhancers, and termination elements) in an expression vector, e.g., rAAV vector.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (polyA) sequences) which flank a sequence, such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for vector packaging into a rAAV particle. In various embodiments, a filler/stuffer nucleic acid sequence is an untranslated (non-protein encoding) segment of nucleic acid. For a nucleic acid sequence less than 4.7 Kb, the filler or stuffer polynucleotide sequence has a length that when combined (e.g., inserted into a vector) with the sequence has a total length between about 3.0-5.5 Kb, or between about 4.0-5.0 Kb, or between about 4.3-4.8 Kb.

A “therapeutic protein” in one embodiment is a peptide or protein that may alleviate or reduce symptoms that result from an insufficient amount, absence or defect in a protein in a cell or subject. A “therapeutic” protein encoded by a transgene can confer a benefit to a subject, e.g., to correct a genetic defect, to correct a gene (expression or functional) deficiency, etc.

Non-limiting examples of heterologous nucleic acids encoding gene products (e.g., therapeutic proteins) which are useful in accordance with the invention include those that may be used in the treatment of a disease or disorder including, but not limited to, “hemostasis” or blood clotting disorders such as hemophilia A, hemophilia A patients with inhibitory antibodies, hemophilia B, deficiencies in coagulation Factors, VII, VIII, IX and X, XI, V, XII, II, von Willebrand factor, combined FV/FVIII deficiency, thalassemia, vitamin K epoxide reductase Cl deficiency, gamma-carboxylase deficiency; anemia, bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e. FXa inhibitors); and platelet disorders such as, Bernard Soulier syndrome, Glanzman thromblastemia, and storage pool deficiency.

Nucleic acid molecules, vectors such as cloning, expression vectors (e.g., vector genomes) and plasmids, may be prepared using recombinant DNA technology methods. The availability of nucleotide sequence information enables preparation of nucleic acid molecules by a variety of means. For example, a heterologous nucleic acid encoding Factor IX (FIX) comprising a vector or plasmid can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane.

With respect to protein, the term “isolated protein” or “isolated and purified protein” is sometimes used herein. This term refers primarily to a protein produced by expression of a nucleic acid molecule. Alternatively, this term may refer to a protein which has been sufficiently separated from other proteins with which it would naturally be associated, so as to exist in “substantially pure” form.

The term “isolated” does not exclude combinations produced by the hand of man, for example, a recombinant rAAV and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or about 90-99% by weight, of the compound of interest. Purity is measured by methods appropriate for the compound of interest (e.g. chromatographic methods, agarose or polyacrylamide gel electrophoresis, HPLC analysis, and the like).

The phrase “consisting essentially of” when referring to a particular nucleotide sequence or amino acid sequence means a sequence having the properties of a given sequence. For example, when used in reference to an amino acid sequence, the phrase includes the sequence per se and molecular modifications that would not affect the basic and novel characteristics of the sequence.

Methods that are known in the art for generating rAAV virions: for example, transfection using AAV vector and AAV helper sequences in conjunction with coinfection with one AAV helper viruses (e.g., adenovirus, herpesvirus, or vaccinia virus) or transfection with a recombinant AAV vector, an AAV helper vector, and an accessory function vector. Non-limiting methods for generating rAAV virions are described, for example, in U.S. Pat. Nos. 6,001,650 and 6,004,797, International Application PCT/US16/64414 (published as WO 2017/096039) and U.S. Provisional Application Nos. 62/516,432 and 62/531,626. Following recombinant rAAV vector production (i.e. vector generation in cell culture systems), rAAV virions can be obtained from the host cells and cell culture supernatant and purified as set forth herein.

As an initial step, typically host cells that produce the rAAV virions can be harvested, optionally in combination with harvesting cell culture supernatant (medium) in which the host cells (suspension or adherent) producing rAAV virions have been cultured. In methods herein, the harvested cells and optionally cell culture supernatant may be used as is, as appropriate, or concentrated. Further, if infection is employed to express accessory functions, residual helper virus can be inactivated. For example, adenovirus can be inactivated by heating to temperatures of approximately 60° C. for, e.g., 20 minutes or more, which inactivates only the helper virus since AAV is heat stable while the helper adenovirus is heat labile.

Cells and/or supernatant of the harvest are lysed by disrupting the cells, for example, by chemical or physical means, such as detergent, microfluidization and/or homogenization, to release the rAAV particles. Concurrently during cell lysis or subsequently after cell lysis, a nuclease such as benzonase may be added to degrade contaminating DNA. Typically, the resulting lysate is clarified to remove cell debris, such as filtering, centrifuging, to render a clarified cell lysate. In a particular example, lysate is filtered with a micron diameter pore size filter (such as a 0.1-10.0 μm pore size filter, for example, a 0.45 μm and/or pore size 0.2 μm filter), to produce a clarified lysate.

The lysate (optionally clarified) contains AAV particles (bona fide rAAV vectors, and AAV empty capsids) and AAV vector production/process related impurities, such as soluble cellular components from the host cells that can include, inter alia, cellular proteins, lipids, and/or nucleic acids, and cell culture medium components. The optionally clarified lysate is then subjected to additional purification steps to purify AAV particles (including bona fide rAAV vectors) from impurities using chromatography. Clarified lysate may be diluted or concentrated with an appropriate buffer prior to the first step of chromatography.

As disclosed herein, after cell lysis, optional clarifying, and optional dilution or concentration, a plurality of sequential chromatography steps are used to purify rAAV particles. Such methods typically exclude a step of cesium chloride gradient ultracentrifugation.

As disclosed herein, a first chromatography step may be cation exchange chromatography or anion exchange chromatography. If the first chromatography step is cation exchange chromatography the second chromatography step can be anion exchange chromatography or size exclusion chromatography (SEC). Thus, in one rAAV purification method, purification is via cation exchange chromatography, followed by purification via anion exchange chromatography.

Alternatively, if the first chromatography step is cation exchange chromatography the second chromatography step can be size exclusion chromatography (SEC). Thus, in another rAAV purification method, purification is via cation exchange chromatography, followed by purification via size exclusion chromatography (SEC).

As also disclosed herein, a first chromatography step may be affinity chromatography. If the first chromatography step is affinity chromatography the second chromatography step can be anion exchange chromatography. Thus, in a further rAAV purification method, purification is via affinity chromatography, followed by purification via anion exchange chromatography.

Optionally, a third chromatography can be added to the foregoing chromatography steps. Typically, the optional third chromatography step follows cation exchange, anion exchange, size exclusion or affinity chromatography.

Thus, in an additional rAAV purification method, purification is via cation exchange chromatography, followed by purification via anion exchange chromatography, followed by purification via size exclusion chromatography (SEC). And, in a still further rAAV purification method, purification is via cation exchange chromatography, followed by purification via size exclusion chromatography (SEC), followed by purification via anion exchange chromatography.

In yet an additional rAAV purification method, purification is via affinity chromatography, followed by purification via anion exchange chromatography, followed by purification via size exclusion chromatography (SEC). In yet another rAAV purification method, purification is via affinity chromatography, followed by purification via size exclusion chromatography (SEC), followed by purification via anion exchange chromatography.

Cation exchange chromatography functions to separate the AAV particles from cellular and other components present in the clarified lysate and/or column eluate from the size exclusion chromatography. Examples of strong cation exchange resins capable of binding rAAV particles over a wide pH range include, without limitation, any sulfonic acid based resins as indicated by the presence of the sulfonate functional group, including aryl and alkyl substituted sulfonates, such as sulfopropyl or sulfoethyl resins. Representative matrices include but are not limited to POROS HS, POROS HS 50, POROS XS, POROS SP, and POROS S (strong cation exchangers available from Thermo Fisher Scientific, Inc., Waltham, Mass.). Additional examples include Capto S, Capto S ImpAct, Capto S ImpRes (strong cation exchangers available from GE Healthcare, Marlborough, Mass.), and commercial DOWEX®, AMBERLITE®, and AMBERLYST® families of resins available from Aldrich Chemical Company (Milliwaukee, Wis.). Weak cation exchange resins include, without limitation any carboxylic acid based resins. Exemplary cation exchange resins also include carboxymethyl (CM), phospho (based on the phosphate functional group), methyl sulfonate (S) and sulfopropyl (SP) resins.

Anion exchange chromatography functions to separate AAV particles from proteins, cellular and other components present in the clarified lysate and/or column eluate from the size exclusion chromatography. Anion exchange chromatography can also be used to control the amount of AAV empty capsids in the eluate. For example, the anion exchange column having rAAV vector bound thereto can be washed with NaCl at a modest concentration (e.g., about 100-125 mM, such as 110-115 mM) and a portion of the empty capsids can be eluted in the flowthrough without substantial elution of the rAAV vectors. Subsequently, rAAV vector bound to the anion exchange column can be eluted using NaCl at a higher concentration (e.g., about 130-300 mM Nacl), thereby producing a column eluate with reduced or depleted amounts of AAV empty capsids and proportionally increased amounts of rAAV.

Exemplary anion exchange resins include, without limitation, those based on polyamine resins and other resins. Examples of strong anion exchange resins include those based generally on the quaternized nitrogen atom including, without limitation, quaternary ammonium salt resins such as trialkylbenzyl ammonium resins. Suitable exchange chromatography include without limitation, MACRO PREP Q (strong anion-exchanger available from BioRad, Hercules, Calif.); UNOSPHERE Q (strong anion-exchanger available from BioRad, Hercules, Calif.); POROS 50HQ (strong anion-exchanger available from Applied Biosystems, Foster City, Calif.); POROS XQ (strong anion-exchanger available from Applied Biosystems, Foster City, Calif.); POROS 50D (weak anion-exchanger available from Applied Biosystems, Foster City, Calif.); POROS 50PI (weak anion-exchanger available from Applied Biosystems, Foster City, Calif.); Capto Q, Capto XQ, Capto Q ImpRes, and SOURCE 30Q (strong anion-exchanger available from GE healthcare, Marlborough, Mass.); DEAE SEPHAROSE (weak anion-exchanger available from Amersham Biosciences, Piscataway, N.J.); Q SEPHAROSE (strong anion-exchanger available from Amersham Biosciences, Piscataway, N.J.). Additional exemplary anion exchange resins include aminoethyl (AE), diethylaminoethyl (DEAE), diethylaminopropyl (DEPE) and quaternary amino ethyl (QAE).

Chromatography medium such as cation exchange, anion exchange, size exclusion and affinity can be equilibrated, washed and eluted with various buffers under various conditions such as pH, and buffer volumes. The following is intended to describe particular non-limiting examples, but is not intended to limit the invention.

Cation exchange chromatography may be equilibrated using standard buffers and according to the manufacturer's specifications. For example, chromatography media can be equilibrated with a phosphate buffer, at 5 to 100 mM, or 10-50 mM, such as 10-30 mM, and sodium chloride. After equilibration, sample is then loaded. Subsequently, the chromatography media is washed at least once, or more, e.g., 2-10 times. Elution from the chromatography media is by way of a high salt buffer, at least once, but elution may be 2 or more times with the same or a higher salt buffer.

Typical equilibration buffers and solutions for washes and elutions for cation exchange chromatography are at an appropriate pH, of from about pH 3 to pH 8, more typically from about pH 4 to pH 7.5, such as pH 6.0-6.5, 6.5-7.0, 7.0-7.5. or any pH at or between the stated ranges such as, 7.0, 7.1, 7.2, 7.3 or 7.4.

Appropriate equilibration buffers and solutions for washes and elutions for cation exchange columns are known in the art and are generally anionic. Such buffers include, without limitation, buffers with the following buffer ions: phosphate, acetate, citrate, borate, or sulfate.

In one embodiment, the cation exchange chromatography media is first equilibrated, sample applied, and washed with a low salt concentration, e.g., 10-150 mM of NaCl, such as 10, 20, 25, 30, 35, 40, 45, 50, 55, 60, 60-125 mM, or any concentration at or within these ranges, such as, 100 mM.

Following a first wash, the chromatography media may be treated with a higher salt concentration in order to elute impurities, such as a higher NaCl concentration, or with another buffer with a greater ionic strength. After additional impurities are eluted from the column, to elute rAAV particles, the ionic strength of the buffer may be increased using a salt, such as NaCl, KCl, sulfate, formate or acetate, and recovered. In one embodiment, elution is with a high salt concentration, e.g., 200-500 mM of NaCl, or any concentration at or within these ranges, such as 250 mM, 300 mM, 350 mM, or 400 mM.

Additional components can be included in the equilibration buffers and solutions for washes and elutions. For example, a wash buffer for cation exchange chromatography can include an anionic surfactant such as sarkosyl (e.g., 1-10 mM), a wash buffer for anion exchange chromatography can include a cationic surfactant such as Dodecyltrimethylammonium chloride (e.g., 1-10 mM).

Typical equilibration buffers and solutions for washes and elutions for anion exchange chromatography an appropriate at a pH of from about pH 7.5 to pH 12, more typically from about pH 8.0 to pH 10, and even more typically from about pH 8.0 to pH 9.0, such as pH 8.1, 8.2, 8.3, 8.4, 8.5, 8.6, 8.7, 8.8, 8.9 or 9.0.

Appropriate equilibration buffers and solutions for washes and elutions for anion exchange columns are generally cationic or zwitterionic in nature. Such buffers include, without limitation, buffers with the following buffer agents: N-methylpiperazine; piperazine; Bis-Tris; Bis-Tris propane; Triethanolamine; Tris; N-methyldiethanolamine; 1,3-diaminopropane; ethanolamine; acetic acid, and the like. To elute the sample, the ionic strength of the starting buffer is increased using a salt, such as NaCl, KCl, sulfate, formate or acetate. Such equilibration buffers and solutions for washes and elutions can have the foregoing buffering agents from about 5-100 mM, more typically from about 10-50 mM.

In one embodiment, the anion exchange chromatography media is first equilibrated, sample applied, and washed with a low salt concentration, e.g., 50-150 mM of NaCl, such as 50-60, 60-70, 70-80, 80-90, 90-100, 100-100 mM, or any concentration at or within these ranges. Following a first wash, the chromatography media may be treated with a higher salt concentration in order to elute impurities such as AAV empty capsids, such as a higher NaCl concentration, or with another buffer with a greater ionic strength. One example for use as the second buffer is a Tris-based buffer with a NaCl concentration of about 110 mM-125 mM, or any concentration at or within these stated ranges.

After additional impurities are eluted from the column, the AAV particles can be recovered by elution with a higher concentration of salt. One example for an elution buffer is a Tris-based buffer with a NaCl concentration of 125 mM or greater, such as 125-150 mM, 150-200 mM or 200-250 MM NaCl, or any concentration at or within these stated ranges.

In the anion exchange chromatography media wash solutions, polyethylene glycol (PEG) may be included. This is referred to as polyethylene glycol (PEG) modulated column chromatography. PEG wash solutions can be applied to the anion exchange chromatography media prior to elution of AAV vector particles.

Typically PEG in such wash solutions has an average molecular weight in a range of about 1,000 to 80,000 g/mol, inclusive. Typical amounts of PEG in such wash solutions range from about 0.1% to about 20% PEG or any amount at or within these stated ranges, or from about 1% to about 10% PEG or any amount at or within these stated ranges.

Size-exclusion chromatography (SEC) media may be equilibrated using standard buffers and according to the manufacturer's specifications. For example, chromatography media can be equilibrated with a phosphate buffer, for example, at about 1-5 mM, 5-50 mM, or 5-25 mM, and NaCl, for example, at about 50-100 mM, 100-150 mM, 150-200 mM, 200-250 mM, 250-300 mM, or 300-400 mM, or any amount at or within these stated ranges.

After equilibration, sample is then loaded. Subsequently, the flow through containing the rAAV particles is recovered. Additional volumes of buffer (e.g., phosphate buffer), based upon the amount of chromatography media and/or column size, can be added for rAAV particle recovery.

In particular embodiments, size exclusion chromatography media has a separation range (Molecular weight) between about 10,000 and 600,000, inclusive. Particular resins (media) appropriate for size exclusion chromatography include without limitation particles or beads of porous cellulose, crosslinked agarose (Sepharose, GE Healthcare, Marlborough, Mass.), crosslinked dextran (Sephadex, GE Healthcare, Marlborough, Mass.), styrene-divinylbenzene (Dianon HP-20), polyacrylamide (Bio Gel), methacrylic (Toyopearl), and controlled pore glass.

Affinity columns are typically composed of a ligand linked or conjugated to a substrate. Particular examples of ligands include AAV binding antibodies. Such substrates include sepharose and other materials typically used in such affinity purification applications and can be made or are commercially available (e.g., AVB Sepharose™ High Performance, GE Healthcare, Marlborough, Mass.).

Appropriate equilibration buffers and solutions for washes and elutions for affinity columns are typically Tris or acetate based. For example, affinity chromatography media can be equilibrated with a Tris buffer, for example, at about 1-5 mM, 5-50 mM, or 5-20 mM, and NaCl, for example, at about 50-100 mM, 100-150 mM, 150-200 mM, 200-250 mM, 250-300 mM, or any amount at or within these stated ranges.

Typical equilibration buffers for affinity chromatography is a pH of from about pH 7.5 to pH 9.0, more typically from about pH 8.0 to pH 8.5, and even more typically a pH such as pH 8.0, 8.1, 8.2, 8.3, 8.4, or 8.5.

After equilibration, sample is then loaded. Subsequently, the rAAV particles are eluted from the for affinity column by reducing pH of the buffer to less than 7.0. Elution buffers may be acetate based and typically pH is less than 5.0, more typically less than 4.0, such as less than 3.0, more specifically between about 2.0 and 3.0, or any pH at or within these stated ranges.

Volumes of buffer for equilibration, washing and elution can be based upon the amount of chromatography media and/or column size to achieve rAAV particle recovery. Typical volumes are 1-10 column volumes.

Column eluate is/are collected following the elution(s)/flow through from each of the chromatography steps. AAV can be detected in the fractions using standard techniques, such as monitoring UV absorption at 260 and 280 nm.

The use of cation or anion exchange chromatography media, the nature of the media used (i.e. strong or weak ion exchangers) and conditions of salt concentration, buffer used, and pH, can vary based upon the AAV capsid (i.e. AAV capsid serotype or pseudotype). While AAV capsid structure typically share features such as size and shape, capsids may have different amino acid sequences that result in subtle differences of molecular topology and surface charge distribution. Thus, capsid sequence variants are expected to be amenable to purification by the methods of the invention, and relevant methods can be determined in a systematic manner using chromatography media and buffer screening studies, to determine if different conditions will be used for a AAV capsid variant for rAAV particle purification.

Eluates comprising rAAV particles from any of the cation exchange, anion exchange, size exclusion, and/or affinity chromatography steps as described herein can, if desired, be efficiently concentrated by ultrafiltration/diafiltration. Reduction in volume can be controlled by the skilled artisan. In particular non-limiting examples the reduction in volume achieved is between abut 1-30 fold, inclusive. Thus, a 1-fold reduction reduces the volume by half, e.g., 1000 ml is concentrated to 500 mL. A 10 fold reduction reduces the volume by a factor of 10, e.g., 2000 ml is concentrated to 200 mL. A 20 fold reduction reduces the volume by a factor of 20, e.g., 2000 ml is concentrated to 100 mL. A 30 fold reduction reduces the volume by a factor of 30, e.g., 2000 ml is concentrated to 66.67 mL.

A non-limiting example of ultrafiltration/diafiltration is tangential flow filtration (TFF). For example, a hollow fiber membrane with a nominal pore size corresponding to a 100 kDa molecular weight cutoff, so that large amounts of AAV vector can be prepared when present in larger volumes of eluate.

The cell lysate and column eluates comprising rAAV particles from any of the cation exchange, anion exchange, size exclusion, or affinity chromatography steps as described herein can, if desired, be diluted. Typical dilutions range from 25-100%, 1-2 fold, 2-5 fold or any volume or amount at or within these stated ranges.

Methods of the invention achieve substantial recovery of rAAV particles. For example, methods of the invention achieve recovery of rAAV particles of approximately 40-70% of the total rAAV vector particles from the host cells and host cell culture supernatant harvested. In another example, rAAV particles are present in the final (e.g., third column) eluate at a concentration of about 100 mg/mL. rAAV vector particles may be present in the final (e.g., third column) eluate at a concentration of about 10¹⁰-10¹¹ particles per mL, or more, 10¹¹-10¹² particles per mL, 10¹²-10¹³ particles per mL.

Alternatively, if rAAV vector particle concentrations are less, purified rAAV particles can be concentrated. For example, purified AAV particles can be concentrated by ultrafiltration/diafiltration (e.g., TFF). If higher concentrations of vector are desired, purified AAV particles can be concentrated to 10¹²-10¹³ particles per mL, or more, 10¹³-10¹⁴ particles per mL or more, by ultrafiltration/diafiltration (e.g., TFF), or even higher.

In other embodiments, rAAV particles with packaged genomes (i.e., bona fide rAAV vector particles) are “substantially free of “AAV-encapsidated nucleic acid impurities” when at least about 30% or more of the virions present are rAAV particles with packaged genomes (i.e., bona fide rAAV vector particles). Production of rAAV particles with packaged genomes (i.e., bona fide rAAV vector particles) substantially free of AAV-encapsidated nucleic acid impurities can be from about 40% to about 20% or less, about 20% to about 10%, or less, about 10% to about 5% or less, about 5% to about 1% or less than 1% or less of the product comprises AAV-encapsidated nucleic acid impurities.

Methods to determine infectious titer of AAV vector containing a transgene are known in the art (See, e.g., Zhen et al., (2004) Hum. Gene Ther. (2004) 15:709). Methods for assaying for empty capsids and AAV vector particles with packaged genomes are known (See, e.g., Grimm et al., Gene Therapy (1999) 6:1322-1330; Sommer et al., Molec. Ther. (2003) 7:122-128).

To determine the presence or amount of degraded/denatured capsid, purified AAV can be subjected to SDS-polyacrylamide gel electrophoresis, consisting of any gel capable of separating the three capsid proteins, for example, a gradient gel, then running the gel until sample is separated, and blotting the gel onto nylon or nitrocellulose membranes. Anti-AAV capsid antibodies are then used as primary antibodies that bind to denatured capsid proteins (See, e.g., Wobus et al., J. Virol. (2000) 74:9281-9293). A secondary antibody that binds to the primary antibody contains a means for detecting the primary antibody. Binding between the primary and secondary antibodies is detected semi-quantitatively to determine the amount of capsids. Another method would be analytical HPLC with a SEC column or analytical ultracentrifuge.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features (e.g., nucleic acid sequences, vectors, rAAV vectors, etc.) are an example of a genus of equivalent or similar features.

As used herein, the singular forms “a”, “and,” and “the” include plural referents unless the context clearly indicates otherwise. Thus, for example, reference to “an AAV vector,” or “AAV particle,” includes a plurality of such AAV vectors and AAV particles, and reference to “a cell” or “host cell” includes a plurality of cells and host cells.

The term “about” as used herein means values that are within 10% (plus or minus) of a reference value.

As used herein, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges are inclusive. Further, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed.

### EXAMPLES

### Example 1

### Exemplary First Column, Cation Exchange or Affinity

1.1. Cation, Poros50 HS (or Poros XS from ThermoFisher). Precondition, equilibration, load material, wash and elute
   1.1.1. Exemplary Buffer list
      1.1.1.1. Buffer, pH, conductivity
         a) Precondition: water for injection
         b) 3× Dilution buffer: 60 mM Na-Phosphate, pH 7.3
         c) Equilibration buffer: 20 mM Na-Phosphate, pH 7.3, 100 mM NaCl
         d) Washing buffer 1 & 3: 20 mM Na-Phosphate, pH 7.3, 100 mM NaCl
         e) Washing buffer 2: 20 mM Na-Phosphate, pH 7.3, 100 mM NaCl, surfactant (5 mM) such as Sarkosyl
         f) Elution buffer: 20 mM Na-Phosphate, pH 7.3, 300 mM NaCl (can be greater than 300 mM, such as 300-400, e.g. 400 mM NaCl).
         g) Collect eluate based on either elution buffer volume (e.g., collect the eluate after pre-peak volume which is pre-determined) or an increase of O.D. A₂₈₀, such as a 1 mAu increase of the baseline O.D. A₂₈₀.
         h) Column stripping solution: high salt, such as 1M NaCl
         i) Sanitizing solution: 1M NaCl, 1N NaOH
         j) Storage solution: about 22.5% Ethanol (e.g. 18-22.5%)
   1.1.2. Exemplary Column Prep
      1.1.2.1. Pre-condition Downflow (4 CVs) WFI
      1.1.2.2. Equilibration Downflow (4 CVs) Equilibration Buffer
   1.1.3. Exemplary Purification
      1.1.3.1. Load Material Downflow (25 CVs) Sample
      1.1.3.2. Wash 1 Downflow (4 CVs) Wash 1 Buffer
      1.1.3.3. Wash 2 Downflow (6 CVs) Wash 2 Buffer
      1.1.3.4. Wash 3 Downflow (8 CVs) Wash 3 Buffer
   1.1.4. Exemplary Block (Wash) volume(s) determined by scale, column size
   1.1.5. Binding capacity: 2.5-3× loading (Poros50 HS) with or without UF (ultrafiltration)/DF (diafiltration).
   1.1.6. Exemplary Flow rate: 150 cm/h-300 cm/h
   1.1.7. Load capacity: 10 L or more
   1.1.8. Expected Recovery: 60-90%
1.2. Affinity, AVB-Sepharose HP
   1.2.1. Buffer list
      1.2.1.1. Buffer (pH, conductivity) Each block volume
      1.2.1.2. Exemplary buffers/solutions
         a) Equilibration buffer: 20 mM Tris, pH 8.0, 250 mM NaCl
         b) Elution buffer: 10 mM Na-Acetate, pH 2.5, 250 mM NaCl
         c) Neutralization buffer: 1M Tris-Cl, pH 10.5
         d) Sanitization solution: PAB (120 mM phosphoric acid, 167 mM acetic acid, 2.2% benzyl alcohol)
         e) Storage solution: about 22.5% Ethanol (18-22.5%)
   1.2.2. Exemplary Flow rate: about 150 cm/h or more
   1.2.3. Expected Recovery: 70-90% (AAV virion capsid), 15-70% (vector genomes)

### Example 2

### Exemplary Second Column, Anion Exchange or Size Exclusion

1.3. Anion, Poros 50HQ (Poros XQ from ThermoFisher). Precondition, equilibration, load material, wash and elute: used for dual function (Further purification & Empty/Full vector ratio control)
   1.3.1. Exemplary Buffer list
      1.3.1.1. Buffer, pH, conductivity, Each block volume
         a) Precondition: water for injection
         b) 3× (or 4×) dilution buffer: about 60 mM (or about 80 mM) Tris, pH 8.5 (e.g., after dilution, loading material is in the range of 10-50 mM Tris, pH 8.0-8.5, 100 mM NaCl)
         c) Equilibration buffer (and wash 1 buffer): 20 mM Tris, pH 8.5, 100 mM NaCl
         d) Wash 2 buffer (for AAV empty capsid removal): 20 mM Tris, pH 8.5, 115 mM NaCl
         e) Elution buffer: 20 mM Tris, pH8.5, NaCl≥120 mM, such as 200 mM NaCl
         f) Column stripping buffer: 1M NaCl
         g) Sanitization solution: 1N NaOH
         h) Storage solution: about 22.5% Ethanol (e.g. 18-22.5%)
   1.3.2. Exemplary Column Prep
      1.3.2.1. Pre-Condition Downflow (5 CVs) WFI
      1.3.2.2. Equilibration Downflow (4 CVs) Equilibration Buffer
   1.3.3. Exemplary Purification
      1.3.3.1. Load Material Downflow (50 CVs) CEX eluate & diluate
      1.3.3.2. Wash 1 Downflow (5 CVs) Equilibration Buffer
      1.3.3.3. Elution 1 (empty capsid removal) Downflow (3 CVs) Elution Buffer 1
      1.3.3.4. Elution 2 (full AAV vector recovery) Downflow (3 CVs) Elution Buffer 2
   1.3.4. Exemplary Flow rate: 150 cm/h-300 cm/h
1.4. Size exclusion, SEC (e.g., Superdex 200 prep grade from GE healthcare), optional
   1.4.1. Exemplary Buffer list
      1.4.1.1. Buffer, pH, conductivity, Each block volume
         a) Precondition: water for injection
         b) Equilibration washing & elution buffers: 10 mM Na-Phosphate, pH 7.2, 150-300 mM NaCl (higher NaCl, such as 300 mM should improve AAV vector (vg) recovery.
         c) Collect eluate based on either elution buffer volume (e.g., collect the eluate after pre-peak volume which is pre-determined) or an increase of O.D. A₂₈₀, such as a 1 mAu increase of the baseline O.D. A₂₈₀.
         d) Sanitization solution: 0.5N NaOH
         e) Storage solution: 22.5% Ethanol (e.g. 18-22.5%)
   1.4.2. Exemplary Column Prep
      1.4.2.1. Pre-condition Downflow (2 CVs) WFI
      1.4.2.2. Equilibration Downflow (2 CVs) Equilibration Buffer (PBS 300)
   1.4.3. Exemplary Purification
      1.4.3.1. Load Material Downflow (0.05 CVs)
      1.4.3.2. Elution Downflow (1.5 CVs) Equilibration Buffer (dPBS)
      1.4.4. Exemplary Loading capacity: ≤5% of column volume
      1.4.5. Exemplary Flow rate: 45 cm/h
      1.4.6. Collect eluate based on either elution buffer volume or O.D. A₂₈₀

### Example 3

Exemplary Third column, size exclusion or anion exchange. Third column is optional, and may not be needed when affinity column (e.g., AVB-Sepharose HP) is the first column. The third column used is also based on the second column used (SEC>HQ, or HQ>SEC, etc.).
1.5. Size exclusion, SEC (e.g., Superdex 200 prep grade from GE healthcare), optional
   1.5.1. Exemplary Buffer list
      1.5.1.1. Buffer, pH, conductivity, Each block volume
         a) Precondition: water for injection
         b) Equilibration and running buffer: 10 mM Na—P, pH 7.2, 150-300 mM NaCl (higher NaCl, such as 300 mM should improve AAV vector (vg) recovery
         c) Collect eluate based on either elution buffer volume (e.g., collect the eluate after pre-peak volume which is pre-determined) or an increase of O.D. A₂₈₀, such as a 1 mAu increase of the baseline O.D. A₂₈₀.
         d) Sanitization solution: 0.5N NaOH
         e) Storage solution: 22.5% Ethanol
   1.5.2. Exemplary Column Prep
      1.5.2.1. Pre-condition Downflow (2 CVs) WFI
      1.5.2.2. Equilibration Downflow (2 CVs) Equilibration Buffer (PBS 300)
   1.5.3. Exemplary Purification
      1.5.3.1. Load Material Downflow (0.05 CVs)
      1.5.3.2. Elution Downflow (1.5 CVs) Equilibration Buffer (dPBS)
   1.5.4. Exemplary Loading capacity: ≤5% of column volume
   1.5.5. Exemplary Flow rate: 45 cm/h
   1.5.6. Collect eluate based either on elution buffer volume or O.D. A₂₈₀
1.6. Anion, Poros 50HQ (Poros XQ from ThermoFisher) for dual function (Further rAAV purification & Empty/Full rAAV vector ratio control). Precondition, equilibration, load material, wash and elute
   1.6.1. Exemplary Buffer list
      1.6.1.1. Buffer, pH, conductivity, Each block volume
         a) Precondition: water for injection
         b) 3× (or 4×) dilution buffer: 60 mM (or 80 mM) Tris, pH 8.5 (e.g., after dilution, loading material is in the range of 10-50 mmM Tris, pH 8.0-8.5, 100 mM NaCl)
         c) Equilibration buffer (and wash 1 buffer): 20 mM Tris, pH 8.5, 100 mM NaCl
         d) Wash 2 buffer (for AAV empty capsid removal): 20 mM Tris, pH 8.5, 115 mM NaCl
         e) Elution buffer: 20 mM Tris, pH8.5, NaCl≥120 mM, such as 200 mM NaCl
         f) Column stripping buffer: 1M NaCl
         g) Sanitization solution: 1N NaOH
         h) Storage solution: 22.5% Ethanol (e.g. 18-22.5%)
   1.6.2. Exemplary Column Prep
      1.6.2.1. Pre-Condition Downflow (5 CVs) WFI
      1.6.2.2. Equilibration Downflow (4 CVs) Equilibration Buffer
   1.6.3. Exemplary Purification
      1.6.3.1. Load Material Downflow (50 CVs) CEX eluate & diluate
      1.6.3.2. Wash 1 Downflow (5 CVs) Equilibration Buffer
      1.6.3.3. Elution 1 (empty capsid removal) Downflow (3 CVs) Elution Buffer 1
      1.6.3.4. Elution 2 (full AAV vector recovery) Downflow (3 CVs) Elution Buffer 2
   1.6.4. Exemplary Flow rate: 150 cm/h-300 cm/h

### Example 4

Exemplary cell lysis and preparation prior to column purification.
1. Cell lysis, chemical or physical
   1.1 Exemplary Chemical lysis method
      1.6.5. Triton X-100 or equivalent non-ionic surfactant
      i. Final concentration, 0.1%-1%
      ii. Incubation time, up to 1 hour
      iii. Agitation speed of the impeller is typically about 400-600 rpm (or more)
      iv. Incubation temperature is about 25-37° C. (e.g., 37° C.)
      1.6.6. Benzonase
      i. Final concentration, ≥50 U/mL, such as 100 U/mL
      ii. Treatment can be simultaneously or sequentially with surfactant.
      iii. MgCl₂ concentration is 1.0-5.0 mM (e.g., 2 mM)
      1.1.3. Additional salt to help recover AAV vector during or following filtration step
      II. Final concentration of NaCl is 200-400 mM (e.g., 300 mM)
      III. NaCl, other salt equivalents to NaCl can be used
1.7. Exemplary Physical lysis method (Microfluidization, homogenization, etc.)
   1.7.1. Operation condition (based on 10 L adherent cell culture which can be scaled up to larger volumes)
   i. Pressure approximately ≤5,000 psi
   ii. Temperature approximately 18-25° C.
   1.7.2. Pretreated & chasing buffer
   i. Buffer, pH, Conductivity
      All depend on the first column loading condition; in most case, DF buffer is the same or an equivalent buffer used for the equilibration in the first column. If HS is the first column, PBS could be common buffer)
      For benzonase treatment, both pH and conductivity should be within the range of benzonase working condition (e.g., about pH 6.5-8.5, conductivity >15 mS/cm). Typical benzonase amount for DNA digestion is 100-200 U/mL. 2 mM MgCl₂ should be added for proper digestion.
   ii. Volume
      Pretreated volume need to be more than hold-up volume of the system (mostly likely three times of the hold-up volume).
      Chasing volume is 5-10% of the sample volume.
2. Tangential flow filtration (TFF, aka. UF/DF), optional
   2.1. TFF with Hollow fiber cartridges
      2.1.1. In case of 10 L cell culture
      i. TFF is executed before cell lysis
      ii. Filters such as GE hollow fiber cartridges UFP-100-C-9A (1.2 m²) for 10 L
      iii. Capacity. Preliminary data indicates that 20 L should work with same UFP-100-C-9A hollow fiber cartridge.
      iv. TFF before cell lysis, or after cell lysis. TFR before cell lysis
      v. Maximum pressure. TMP. ≥5 psig in case of before cell lysis (5-10 psi in case of after cell lysis)
      vi. Buffer for pretreatment or chasing. (In case of cation HS) as the first column, 20 mM phosphate buffer, pH 7-7.5/100-150 mM NaCl is reasonable buffer in case of (anion HQ) as the first column, 20 mM Tris, pH 7.5-8.5/100-150 mM NaCl is an appropriate buffer.
      2.1.2. In case of ultrafiltration to concentrate intermediate of purified AAV vector before SEC column
      i. TFF to concentrate anion exchange (Poros 50 HQ) eluate to about 5% of SEC column volume
      ii. Material: Poros 50HQ eluate 0.75 column volume
      iii. Configuration: Hollow Fiber UFP-C-100-4MA (in case of 10 L starting volume)
      iv. Flushing Volume (Holdup volume): ˜45 mL
      v. Equilibration Buffer: PBS300 (10 mM Na—P, pH 7.2/300 mM NaCl)
      vi. Sanitization Buffer: 1N NaOH
      vii. Same conditions with adherent cell culture is applicable.
      viii. TFF can be performed before cell lysis or after cell lysis.
      2.1.3. TFF for final formulation and removal of possible impurities with small molecular weight
      i. Material: Poros50 HQ eluate (or SEC vector peak)
      ii. Configuration: Hollow Fiber UFP-C-100-4MA (in case of 10 L starting volume)
      iii. Flushing Volume (Holdup volume): ˜45 mL
      iv. Equilibration Buffer: PBS180 (10 mM Na—P, pH 7.3/180 mM NaCl)
      v. Diafiltration Buffer: PBS180 (10 mM Na—P, pH 7.3/180 mM NaCl)
      vi. Sanitization Buffer: 1N NaOH
      vii. Target Concentration: 1.0E+13 vg/mL
      viii. Diafiltration: Volume 12 times of UF target volume (5.0E12 vg/mL)
   2.2. Alternating Tangential Flow (ATF) or TFF with either spiral-wound membrane module or flat plate module are alternatives to TFF with hollow fiber cartridge
3. Clarification & Filtration
   3.1. Depth filter
      i. Filter options include Clarisolve 20MS (0.5-20 μM), Sartoclear DL series (10-1 μM) or Millistak COHC or DOHC (0.65-8 μM)
      ii. Capacity can be 1 L/25 cm².
      iii. Maximum flow rate, about 250 mL/min/25 cm² (10 mL/min/cm²)
      iv. Maximum operation pressure about ˜32 psig
      v. Conditioning buffer about PBS300 may be good
      vi. Number of filtration steps (one or two, coarse then fine filter): Possibly a single step: coarse filter
   3.2. Millipore SHC (0.5/0.2 μM) or Sartopore 2 (0.45/0.2 μM, Sartorius)
      i. Capacity about 500 mL/500 cm² (0.2% Triton X-100 helps a little bit, 10-20% increase)
      ii. Maximum flow rate about 1000 mL/min/500 cm² (2 mL/min/cm²)
      iii. Maximum operation pressure about 35 psig
      iv. For conditioning buffer, an example is PBS300
      v. Final step the capacity may be limited (required about 55 maxi Sartopore 2 (1.8 m²)); if this is the 2^{nd} filtration step, capacity could be increased
   3.3. In case of exemplary 10 L scale of adherent cell culture
      i. Sartopore 2 MaxiCaps (1.2 m²) can be used for 10 L culture volume
      ii. This same filter can be used with 20 L culture volume.
      iii. Conductivity of the pretreatment & chasing buffer can be about 15-30 mS/cm
      to prevent potential interaction between AAV vector and membrane.

### Example 5

1.0 Desirable Development Criteria for 1.2 L and Larger Volumes of rAAV Vector Production Based on Process Developed for 500-600 ml of rAAV Harvest Volume
   1.1 rAAV Vector (e.g. LK03-FVIII), Harvested from 1.2 L Bioreactor Suspension
   1.2 Purification of rAAV Vector from 1.2 L Bioreactor Suspension Culture

### Example 6

2.0 Harvest of rAAV Vector (e.g. LK03-FVIII) from 1.2 L Bioreactor Suspension Culture
   2.1 Process Flow Diagram
      2.1.1 The following is an exemplary process flow diagram for the Harvest portion of an rAAV vector downstream process:
2.2 Process Flow Description
   2.2.1 The following is an exemplary process flow description for the Harvest portion of the rAAV vector downstream process:
2.3 Parameters Varied during Harvest Operation Process Development Studies
   2.3.1 The following parameters are evaluated for development of the Harvest portion of a rAAV vector downstream process:

### Example 7

As disclosed herein, the lysis methods, column number and type of column can be selected and used in various orders.
4.0 Chromatography Process for 1.2 L Bioreactor Suspension Culture
   4.1 Parameters Varied during rAAV Vector (e.g. LK03-FVIII) Chromatography Process Development Studies
      4.1.1 The following options are developed for the chromatography portion of the rAAV vector downstream process:
   4.2 Exemplary non-limiting column purification orders (A-E):
   A) 1^{st} column, cation (Poros 50HS)→2^{nd} column, anion (e.g., Poros 50HQ)
   B) 1^{st} column, cation (Poros 50HS)→2^{nd} column, anion (e.g., Poros 50HQ)→3^{rd} column, size exclusion (e.g., Superdex 200 PG),
   C) 1st column, cation (Poros 50HS)→2^{nd} column, size exclusion (e.g., Superdex 200 PG)→3^{rd} column, anion (e.g., Poros 50HQ)
   D) 1st column, affinity (AVB Sepharose HP)→2^{nd} column, anion (e.g., Poros 50HQ)→3^{rd} column (optional), size exclusion (e.g., Superdex 200 PG)
   E) 1st column, affinity (AVB Sepharose HP)→2^{nd} column, size exclusion (e.g., Superdex 200 PG)→3^{rd} column (optional), anion (e.g., Poros 50HQ)

### Example 8

Representative AAV capsid (VP1) proteins.

| AAV-SPK VP1 Capsid | |
|---|---|
| (SEQ ID NO: 1) | |
| 1 | MAADGYLPDWLEDNLSEGIREWWDLKPGAPKPKANQQKQDNGRGLVLPGYKYLGPFNGLD |
| 61 | KGEPVNAADAAALEHDKAYDQQLQAGDNPYLRYNHADAEFQERLQEDTSFGGNLGRAVFQ |
| 121 | AKKRVLEPLGLVESPVKTAPGKKRPVEPSPQRSPDSSTGIGKKGQQPAKKRLNFGQTGDS |
| 181 | ESVPDPQPIGEPPAAPSGVGPNTMAAGGGAPMADNNEGADGVGSSSGNWHCDSTWLGDRV |
| 241 | ITTSTRTWALPTYNNHLYKQISNGTSGGSTNDNTYFGYSTPWGYFDFNRFHCHFSPRDWQ |
| 301 | RLINNNWGFRPKRLNFKLFNIQVKEVTQNEGTKTIANNLTSTIQVFTDSEYQLPYVLGSA |
| 361 | HQGCLPPFPADVFMIPQYGYLTLNNGSQAVGRSSFYCLEYFPSQMLRTGNNFEFSYNFED |
| 421 | VPFHSSYAHSQSLDRLMNPLIDQYLYYLSRTQSTGGTAGTQQLLFSQAGPNNMSAQAKNW |
| 481 | LPGPCYRQQRVSTTLSQNNNSNFAWTGATKYHLNGRDSLVNPGVAMATHKDDEERFFPSS |
| 541 | GVLMFGKQGAGKDNVDYSSVMLTSEEEIKTTNPVATEQYGVVADNLQQQNAAPIVGAVNS |
| 601 | QGALPGMVWQNRDVYLQGPIWAKIPHTDGNFHPSPLMGGFGLKHPPPQILIKNTPVPADP |
| 661 | PTTFNQAKLASFITQYSTGQVSVEIEWELQKENSKRWNPEIQYTSNYYKSTNVDFAVNTE |
| 721 | GTYSEPRPIGTRYLTRNL |

AAV-LK03 VP1 Capsid
(SEQ ID NO: 2)
MAADGYLPDWLEDNLSEGIREWWALQPGAPKPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRPL

1. A method for purifying recombinant adeno-associated viral (rAAV) vector particles said method comprising the steps of:
(a) harvesting cells and/or cell culture supernatant comprising rAAV vector particles to produce a harvest;
(b) optionally concentrating said harvest produced in step (a) to produce a concentrated harvest;
(c) lysing said harvest produced in step (a) or said concentrated harvest produced in step (b) to produce a lysate;
(d) treating the lysate produced in step (c) to reduce contaminating nucleic acid in the lysate thereby producing a nucleic acid reduced lysate;
(e) optionally filtering said nucleic acid reduced lysate produced in step (d) to produce a clarified lysate, and optionally diluting said clarified lysate to produce a diluted clarified lysate;
(f) subjecting said nucleic acid reduced lysate in in step (d), clarified lysate in step (e) or diluted clarified lysate produced in step (e) to cation exchange column chromatography to produce a column eluate comprised of rAAV vector particles thereby separating rAAV vector particles from protein impurities or other production/process related impurities, and optionally diluting said column eluate to produce a diluted column eluate;
(g) subjecting said column eluate or said diluted column eluate produced in step (f) to anion exchange chromatography to produce a second column eluate comprised of rAAV vector particles thereby separating rAAV vector particles from protein impurities or production/process related impurities, and optionally concentrating said second column eluate to produce a concentrated second column eluate;
(h) subjecting said second column eluate or said concentrated second column eluate produced in step (g) to size exclusion column chromatography (SEC) to produce a third column eluate comprised of rAAV vector particles thereby separating rAAV vector particles from protein impurities or production/process related impurities, and optionally concentrating said third column eluate to produce a concentrated third column eluate; and
(i) filtering said third column eluate or said concentrated third column eluate produced in step (h) thereby producing purified rAAV vector particles.

2. A method for purifying recombinant adeno-associated viral (rAAV) vector particles said method comprising the steps of:
(a) harvesting cells and/or cell culture supernatant comprising rAAV vector particles to produce a harvest;
(b) optionally concentrating said harvest produced in step (a) to produce a concentrated harvest;
(c) lysing said harvest produced in step (a) or said concentrated harvest produced in step (b) to produce a lysate;
(d) treating the lysate produced in step (c) to reduce contaminating nucleic acid in the lysate thereby producing a nucleic acid reduced lysate;
(e) optionally filtering said nucleic acid reduced lysate produced in step (d) to produce a clarified lysate, and optionally diluting said clarified lysate to produce a diluted clarified lysate;
(f) subjecting said nucleic acid reduced lysate in in step (d), clarified lysate in step (e) or diluted clarified lysate produced in step (e) to cation exchange column chromatography to produce a column eluate comprised of rAAV vector particles thereby separating rAAV vector particles from protein impurities or other production/process related impurities, and optionally concentrating said column eluate to produce a concentrated column eluate;
(g) subjecting said column eluate or said concentrated column eluate produced in step (f) to size exclusion column chromatography (SEC) to produce a second column eluate comprised of rAAV vector particles thereby separating rAAV vector particles from protein impurities or other production/process related impurities, and optionally diluting said second column eluate to produce a diluted second column eluate;
(h) subjecting said second column eluate or said diluted second column eluate produced in step (g) to anion exchange chromatography to produce a third column eluate comprised of rAAV vector particles thereby separating rAAV vector particles from protein impurities production/process related impurities, and optionally diluting said third column eluate to produce a diluted third column eluate; and
(i) filtering said third column eluate or said concentrated third column eluate produced in step (h) thereby producing purified rAAV vector particles.

3. A method for purifying recombinant adeno-associated viral (rAAV) vector particles said method comprising the steps of:
(a) harvesting cells and/or cell culture supernatant comprising rAAV vector particles to produce a harvest;
(b) optionally concentrating said harvest produced in step (a) to produce a concentrated harvest;
(c) lysing said harvest produced in step (a) or said concentrated harvest produced in step (b) to produce a lysate;
(d) treating the lysate produced in step (c) to reduce contaminating nucleic acid in the lysate thereby producing a nucleic acid reduced lysate;
(e) optionally filtering said nucleic acid reduced lysate produced in step (d) to produce a clarified lysate, and optionally diluting said clarified lysate to produce a diluted clarified lysate;
(f) subjecting said nucleic acid reduced lysate in in step (d), clarified lysate in step (e) or diluted clarified lysate produced in step (e) to cation exchange column chromatography to produce a column eluate comprised of rAAV vector particles thereby separating rAAV vector particles from protein impurities or other production/process related impurities, and optionally diluting said column eluate to produce a diluted column eluate;
(g) subjecting said column eluate or said diluted column eluate produced in step (f) to anion exchange chromatography to produce a second column eluate comprised of rAAV vector particles thereby separating rAAV vector particles from production/process related impurities, and optionally concentrating said second column eluate to produce a concentrated second column eluate;
(h) filtering said second column eluate or said concentrated second column eluate produced in step (g) thereby producing purified rAAV vector particles.

4. A method for purifying recombinant adeno-associated viral (rAAV) vector particles said method comprising the steps of:
(a) harvesting cells and/or cell culture supernatant comprising rAAV vector particles to produce a harvest;
(b) optionally concentrating said harvest produced in step (a) to produce a concentrated harvest;
(c) lysing said harvest produced in step (a) or said concentrated harvest produced in step (b) to produce a lysate;
(d) treating the lysate produced in step (c) to reduce contaminating nucleic acid in the lysate thereby producing a nucleic acid reduced lysate;
(e) optionally filtering said nucleic acid reduced lysate produced in step (d) to produce a clarified lysate, and optionally diluting said clarified lysate to produce a diluted clarified lysate;
(f) subjecting said nucleic acid reduced lysate in step (d), or clarified lysate or diluted clarified lysate produced in step (e) to AAV affinity column chromatography to produce a column eluate comprised of rAAV vector particles thereby separating rAAV vector particles from protein impurities or other production/process related impurities, and optionally diluting said column eluate to produce a diluted column eluate;
(g) subjecting said column eluate or said diluted column eluate produced in step (f) to anion exchange chromatography to produce a second column eluate comprised of rAAV vector particles thereby separating rAAV vector particles from protein impurities or other production/process related impurities, and optionally concentrating said second column eluate to produce a concentrated second column eluate;
(h) optionally subjecting said second column eluate or said concentrated second column eluate produced in step (g) to size exclusion column chromatography (SEC) to produce a third column eluate comprised of rAAV vector particles thereby separating rAAV vector particles from protein impurities or other production/process related impurities, and optionally concentrating said third column eluate to produce a concentrated third column eluate; and
(i) filtering said second column eluate or said diluted second column eluate produced in step (g), or filtering said third column eluate or said concentrated third column eluate produced in step (h), thereby producing purified rAAV vector particles.

5. A method for purifying recombinant adeno-associated viral (rAAV) vector particles said method comprising the steps of:
(a) harvesting cells and/or cell culture supernatant comprising rAAV vector particles to produce a harvest;
(b) optionally concentrating said harvest produced in step (a) to produce a concentrated harvest;
(c) lysing said harvest produced in step (a) or said concentrated harvest produced in step (b) to produce a lysate;
(d) treating the lysate produced in step (c) to reduce contaminating nucleic acid in the lysate thereby producing a nucleic acid reduced lysate;
(e) optionally filtering said nucleic acid reduced lysate produced in step (d) to produce a clarified lysate, and optionally diluting said clarified lysate to produce a diluted clarified lysate;
(f) subjecting said nucleic acid reduced lysate in step (d), or clarified lysate or diluted clarified lysate produced in step (e) to AAV affinity column chromatography to produce a column eluate comprised of rAAV vector particles thereby separating rAAV vector particles from protein impurities or other production/process related impurities, and optionally concentrating said column eluate to produce a concentrated column eluate;
(g) subjecting said column eluate or said concentrated column eluate produced in step (f) to size exclusion column chromatography (SEC) to produce a second column eluate comprised of rAAV vector particles thereby separating rAAV vector particles from protein impurities or other production/process related impurities, and optionally diluting said second column eluate to produce a diluted second column eluate;
(h) optionally subjecting said second column eluate or said diluted second column eluate produced in step (g) to anion exchange chromatography to produce a third column eluate comprised of rAAV vector particles thereby separating rAAV vector particles from protein impurities or other production/process related impurities, and optionally diluting said third column eluate to produce a diluted third column eluate; and
(i) filtering said second column eluate or said diluted second column eluate produced in step (g), or filtering said third column eluate or said concentrated third column eluate produced in step (h), thereby producing purified rAAV vector particles.

6. A method according to claim 1, wherein said concentrating of step (b) and/or step (f) and/or step (g) and/or step (h) is via ultrafiltration/diafiltration, optionally or by tangential flow filtration (TFF).

7.-8. (canceled)

9. A method according to claim 1, wherein said lysing of said harvest produced in step (a) or said concentrated harvest produced in step (b) is by physical or chemical means.

10. A method according to claim 9, wherein the physical means comprises microfluidization or homogenization.

11-14. (canceled)

15. A method according to claim 1, wherein said diluting of said clarified lysate of step (e) is with an aqueous buffered phosphate, acetate or Tris solution.

16. A method according to claim 1, wherein said diluting of said column eluate of step (f) or said second column eluate of step (g) is with an aqueous buffered phosphate, acetate or Tris solution.

17-18. (canceled)

19. A method according to claim 1, wherein said rAAV vector particles resulting from step (i) are formulated with a surfactant to produce an AAV vector formulation.

20. A method according to claim 1, wherein said anion exchange column chromatography of step (f), (g) and/or (h) comprises polyethylene glycol (PEG) modulated column chromatography.

21. A method according to claim 20, wherein said anion exchange column chromatography of step (g) and/or (h) comprises washing said column with a PEG solution or an aqueous surfactant solution prior to elution of said rAAV vector particles from the column.

22-24. (canceled)

25. A method according to claim 1, wherein said cation exchange column of step (f) comprises washing said column with a surfactant solution prior to elution of said rAAV vector particles from the column.

26-30. (canceled)

31. A method according to claim 1, wherein said rAAV vector particles are eluted from said anion exchange column of step (f), (g) and/or (h) with an aqueous Tris-Cl/NaCl buffer.

32. (canceled)

33. A method according to claim 1, wherein said anion exchange column of step (f), (g) and/or (h) is washed with an aqueous Tris-Cl/NaCl buffer; or is washed one or more times to reduce the amount of AAV empty capsids in the second or third column eluate.

34-40. (canceled)

41. A method according to claim 1, wherein said rAAV vector particles are eluted from said cation exchange column of step (f) in an aqueous phosphate/NaCl buffer or an aqueous acetate/NaCl buffer.

42-43. (canceled)

44. A method according to claim 1, wherein said anion exchange column of step (f), (g) and/or (h) comprises a quarternary ammonium functional group such as quaternized polythyleneimine.

45. A method according to claim 1, wherein said size exclusion column (SEC) of step (g) and/or (h) has a separation/fractionation range (Molecular weight) from about 10,000 to about 600,000, inclusive.

46. A method according to claim 1, wherein said cation exchange column of step (f) comprises a sulfonic acid or functional group such as sulphopropyl.

47. A method according to claim 3, wherein the AAV affinity column comprises a protein or ligand that binds to AAV capsid protein.

48-49. (canceled)

50. A method according to claim 1, wherein the method excludes a step of cesium chloride gradient ultracentrifugation.

51. A method according to claim 1, wherein said rAAV vector particles comprise a transgene that encodes:
a nucleic acid selected from the group consisting of a siRNA, an antisense molecule, miRNA a ribozyme and a shRNA; or
a gene product selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I and II (IGF-I and IGF-II), TGFβ, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase; or
a gene product selected from the group consisting of thrombopoietin (TPO), interleukins (IL1 through IL-17), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

52-53. (canceled)

54. A method according to claim 1, wherein said rAAV vector particles comprise a transgene encoding a protein useful for correction of in born errors of metabolism selected from the group consisting of carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor V, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, RPE65, H-protein, T-protein, a cystic fibrosis transmembrane regulator (CFTR) sequence, and a dystrophin cDNA sequence.

55. (canceled)

56. A method according to claim 1, wherein the method recovers approximately 50-90% of the total rAAV vector particles from the harvest produced in step (a) or said concentrated harvest produced in step (b).

57. (canceled)

58. A method according to claim 1, wherein steps (c) and (d) are performed substantially concurrently.

59-60. (canceled)

61. A method according to claim 1, wherein said rAAV vector particles comprise a capsid sequence having 70% or more identity to an AAV1, AAV2, AAV3, AAV4, AAV5, AAV6, AAV7, AAV8, AAV9, AAV10, Rh10, Rh74, SEQ ID NO:1 or SEQ ID NO:2 capsid sequence or comprise an ITR sequence having 70% or more identity to an AAV1, AAV2, AAV3, AAV4, AAV5, AAV6, AAV7, AAV8, AAV9, AAV10, Rh10, or Rh74 ITR sequence.

62. (canceled)

63. A method according to claim 1, wherein said cells comprise suspension or adherent cells; or mammalian cells; or HEK-293 cells.

64-67. (canceled)


SEQUENCE LISTING (2 sequences)

SEQ ID NO: 1 - protein, 738 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 2 - protein, 736 aa, Adeno-associated virus
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL